(19) United States
(12) Patent Application Publication
(10) Pub. No.: US 2025/0049601 A1
(43) Pub. Date: Feb. 13, 2025

(54) Delivery Catheter and Methods of Delivery for Aortic ARCH Baroreceptor Hypertension Implants

(71) Applicant: ARCHimedes Vascular, LLC, Birmingham, AL (US)

(72) Inventors: Farrell O. Mendelsohn, Birmingham, AL (US); Steven John, Fremont, CA (US); Craig Bonsignore, Scottsdale, AZ (US)

(73) Assignee: ARCHimedes Vascular, LLC, Birmingham, AL (US)

(21) Appl. No.: 18/929,165

(22) Filed: Oct. 28, 2024

Related U.S. Application Data
(63) Continuation-in-part of application No. PCT/US2023/020608, filed on May 1, 2023.
(60) Provisional application No. 63/594,915, filed on Oct. 31, 2023.
(60) Provisional application No. 63/336,828, filed on Apr. 29, 2022.

(51) Int. Cl. A61F 2/966; A61F 2/86; A61F 2/95

(52) U.S. Cl. CPC A61F 2/966; A61F 2/86; A61F 2/9517; A61F 2250/0097

(57) ABSTRACT

Delivery catheter systems and methods for delivery of hypertension treatment implants are provided. Such catheter devices facilitate delivery and placement of an implant in a target region of an aortic arch of the patient to induce baroreflex. Typically, the catheter includes a catheter shaft, and outer sheath constraining an implant with one or more expandable structures in a collapsed configuration, the sheath being retractable to deploy the implant. The catheter can include markers for precise placement and deployment of the implant along the target region. The catheter can include a retention collar that releasably couples with one or more connectors at proximal ends of one or more tethers extending proximally from the implant to allow precise placement at the target region and/or repositioning or removal of the implant. The target region includes a cylindrical segment of aortic arch between the left common carotid artery and the left subclavian artery.

### CROSS-REFERENCE TO RELATED APPLICATIONS

The present application claims the benefit of priority and is a Non-Provisional of U.S. Provisional Application No. 63/594,915 filed Oct. 31, 2023, and a continuation-in-part of PCT Application No. PCT/US2023/020608 filed May 1, 2023, which claims the benefit of priority of U.S. Provisional Application No. 63/336,828 filed Apr. 29, 2022, the entire contents which are incorporated herein by reference in their entirety for all purposes.

This application is generally related to commonly-owned applications: PCT Patent Application No. PCT/US2023/020612 filed May 1, 2023, entitled “Aortic Arch Baroreceptor Implants for Treatment of Hypertension,” PCT Application No. PCT/US2024/016200 filed Feb. 16, 2024, entitled “Baroreflex Gauge and Mapping Device and Methods of Use,” and U.S. Non-Provisional application Ser. No. ______, [Attorney Docket No. 108743-000150US-1458976] entitled “Aortic Arch Baroreceptor Implants for Treatment of Hypertension,” filed concurrently herewith, the entire contents which are also incorporated herein by reference in their entirety for all purposes.

### FIELD OF THE INVENTION

In one aspect, the invention pertains to delivery of implantable hypertension treatment devices, in particular methods of delivery and delivery catheters.

### BACKGROUND OF THE INVENTION

Hypertension affects one of every two adults in the United States. Yet only 24% of patients have their blood pressure adequately controlled. Hypertension is the leading preventable cause of heart attack, stroke and death. However, a 10 mm Hg drop in blood pressure lowers this cardiovascular risk by 20%.

Pharmacologic therapy has been the mainstay of hypertension treatment for decades, despite the documented poor medical compliance of patients to their antihypertensive medical regimens. In 2004, the landmark trial for a catheter-based medical device (i.e., Symplicity 1) first demonstrated the blood pressure lowering effect of renal denervation by a radiofrequency ablation catheter. Findings from additional clinical trials of renal denervation with radiofrequency and ultrasound energy (i.e., the SPYRAL and RADIANCE trials, respectively) have confirmed these findings, albeit with more modest blood pressure lowering results on the order of 5-10 mm Hg drops in ambulatory recorded systolic blood pressure.

An endovascular implant developed by Vascular Dynamics for hypertension relied on a stent-like device inserted into the carotid artery that lowered blood pressure by stretching the artery wall from the inside and augmenting the carotid baroreflex. In a 2017 study, this carotid baroreflex modulating device lowered ambulatory recorded systolic blood pressure considerably, over twice what had been reported in the renal denervation trials. While clinical results initially appeared promising, with several patients reporting dramatic blood pressure lowerage that persisted two to three years, clinical outcomes were mixed as some patients suffered transient ischemic attacks (TIA), which hindered further trials and subsequent development.

Another challenge arose in regard to deploying the device in the carotid sinus, which was the primary target of the device. Since the carotid sinus carries blood to the brain, any difficulties encountered in this area, for example trauma to arterial tissues during deployment, subsequent dislodgement of the device and/or accumulation of plaques in the region due to the device or trauma, may contribute to TIA or strokes, resulting in adverse or fatal patient outcomes. For clinicians that lack experience with this sensitive region, for example, placing carotid stents, performing procedures in this area may present unnecessary risks to the patient.

Thus, there is a continuing need for hypertension treatment devices and methods that provide the clinical benefits seen in the baroreflex response, yet avoid the considerable drawbacks associated with conventional approaches described above. There is further need for such devices that allow for improved ease and consistency of implantation in order to avoid the noted adverse effects, allow for implantation for a wide variety of clinicians and more reliably provide positive patient outcomes to reduce hypertension long term.

### BRIEF SUMMARY OF THE INVENTION

The invention relates to an implantable hypertension treatment device for deployment within the aortic arch and associated methods of treatment.

In one aspect, the invention pertains to a method of delivering a hypertension treatment implant in a patient. Such methods can include steps of: advancing a delivery catheter carrying an implant for treating hypertension in a distal portion thereof, the implant having one or more expandable structures (e.g., two or more, three, four, etc.) that are interconnected by multiple bridge connectors and being disposed in a collapsed configuration on the delivery catheter to facilitate advancement through the vasculature of the patient; positioning the distal portion of the catheter carrying the implant in the collapsed configuration at a target region within the aortic arch; and deploying the implant within the aortic arch of the patient so that the implant assumes an expanded configuration in which at least a first expandable structure engages an arterial wall along the treatment region to stretch at least a portion of the arterial wall along the target region, thereby triggering a baroreflex response of aortic arch baroreceptors within the target region to reduce blood pressure. The catheter can be advanced along a guidewire positioned within the aortic arch. In some embodiments, the target region is a cylindrical segment wrapping circumferentially around the aortic arch between the left common carotid artery and the left subclavian artery. In some embodiments, the connectors are axially flexible, while in other embodiments, the bridges are helically oriented linear bridges. In some embodiments, the implant includes one or more connectors at a proximal end thereof that releasably couple with a lock element of a delivery shaft to facilitate precise control and/or partial deployment of the implant for repositioning or to allow the implant to be used as a gauge of a physiological response before full deployment. In some embodiments, the connectors are disposed at proximal ends of one or more tethers extending proximally from the implant.

In some embodiments, positioning the distal portion of the catheter can include observing one or more visualization markers disposed on the implant and/or the delivery catheter by visualization techniques, such as fluoroscopy. Positioning the implant at the target region comprises aligning the one or more visualization markers relative to an anatomical feature of the aortic arch and/or secondary branch arteries extending therefrom. In some embodiments, positioning the implant at the target region can entail aligning the one or more visualization markers with a second guidewire disposed within a secondary branch artery extending from the aortic arch, such as the left subclavian artery extending from the aortic arch. In some embodiments, positioning the implant entails aligning the one or more markers relative a second guidewire disposed within the left subclavian artery extending from the aortic arch. In other embodiments, the implant is positioned relative anatomical structures without utilizing a second guidewire.

In some embodiments, the implant is self-expandable from the collapsed configuration to the expanded configuration, where the implant is constrained in the collapsed configuration by a retractable outer sheath of the delivery catheter. For such implants, deploying the implant can entail retracting the outer sheath so as to release the self-expandable implant into the expanded configuration to engage the arterial wall in the target region. In other embodiments, the implant is balloon expandable and is disposed in the collapsed configuration on one or more expandable balloons of the delivery catheter. Deploying the implant can entail expanding the one or more balloons so as to expand the one or more expandable structures into the expanded configuration along the target region. In some embodiments, the implant includes a single expandable structure having a non-circular cross-section. In some embodiments, the implant includes two expandable structures interconnected by multiple flexible connectors that are axially extendable. In some embodiments, the implant includes three expandable structures interconnected by multiple flexible connectors that are axially extendable. In some embodiments, the implant includes three asymmetric structures interconnected by multiple flexible connectors that are axially extendable.

In some embodiments, each of the one or more expandable structures is formed of one or more wires formed in an expandable design (e.g., one or more Nitinol wires formed in a meandering, sinusoidal or zig-zag design to form a circumferential ring or band). In some embodiments, each expandable structure is defined by a single continuous wire. In some such embodiments, the expandable structure can have a substantially circular cross-section. In some embodiments, each expandable structure of the implant includes a plurality of elongated frames having a length greater than a lateral dimension thereof, where the implant is deployed such that the length of the plurality of elongated frames extends in a blood flow direction along the aorta. Each frame can include one or more struts sections that define opposing lateral sides of the frame and are spaced apart a sufficient distance (e.g., 20-40 mm, 30 mm) to reshape and/or to stretch a portion of the arterial wall engaged by the opposing lateral sides of the respective frame by at least 10%, or 20% or more when the implant is deployed. Each of the expandable structures has a length of about 30 mm or greater, and each of the expandable structures can have a greatest lateral dimension between 30 mm and 60 mm, typically between 30 mm and 50. In some embodiments, the expandable structures are expandable rings, and may have designs with peaks and valleys (e.g., sinusoidal, zig-zag). In some embodiments, the entire implant is laser cut from a tube (e.g., Nitinol tube). In some embodiments, the implant is a single expandable structure of a length of about 60 mm or greater so as to extend along a substantial portion of the aortic arch.

In another aspect, the methods can further include withdrawing the delivery catheter and maintaining the implant maintained at the target region to provide long term reduction in blood pressure. Maintaining the position of the implant can entail accommodating a curvature of the aortic arch by multiple expandable structures interconnected by multiple flexible connectors. The accommodation can be partly by expansion of the flexible connectors, where flexible connectors nearer an outer curvature of the aortic arch axially elongate a greater distance than flexible connectors nearer the inner curvature of the aortic arch. Maintaining a position of the deployed implant along the target region long-term can entail deployment of a first expandable structure at the target region and deployment of at least a second expandable structure of the plurality in an adjacent region of the aortic arch upstream from the target region, such that together, engagement of the first and second expandable structures against the arterial wall anchors the implant from dislodgement. The methods can further include inhibiting trauma of the arterial wall during deployment in the aortic arch by proximal and distal atraumatic crowns of each frame, the proximal atraumatic crown connecting proximal ends of the lateral sides and the distal atraumatic crown connecting distal ends of the lateral sides of the respective frame, where the atraumatic crowns comprise an arc of a half circle or less. In some embodiments, the implant includes three expandable structures where the middle expandable structure is disposed at the target location to activate the baroreceptor nerves and the proximal and the proximal and distal expandable structures act as anchors to secure the middle structure at the target location. In such embodiments, the expandable structures can be of the same or differing dimensions. In some embodiments, the implant includes three expandable structures where the middle structure has a lateral dimension that is larger (e.g., 1.3-1.5 times) than the lateral dimension of the proximal and distal expandable structures. This approach allows for even more stretch (e.g., 30% or more) along the target region since proximal and distal structures help transition the arterial walls and inhibit tearing or dissection of the stretched vasculature. At least one of the expandable structures is dimensioned so that when expanded within a target region of the aortic arch, the pair of struts of a respective frame stretch arterial tissues in the target region sufficiently to induce a baroreflex response of baroreceptors in the target region, such as by 10% or more, typically 20% or more, while a major opening between the struts or the open wire design of the structure exposes the arterial wall of the target region to pulsatile blood flow to sustain the baroreflex response long-term.

In another aspect, the invention pertains to a delivery catheter for deploying a hypertension treatment implant in a patient. The delivery catheter can include: a shaft extending between a proximal end and a distal end, the shaft having one or more lumens therethrough; an implant disposed on a distal portion of the shaft in a collapsed configuration, where the implant is convertible between the collapsed configuration and an expanded configuration for engaging the arterial walls within the aortic arch; a retractable outer sheath having a proximal end and a distal end and being disposed over the shaft including the distal end portion having the implant disposed thereon; and a catheter handle disposed at or near the proximal end of the shaft, where the outer sheath is retractable from the catheter handle to facilitate deployment of the implant within the aortic arch. In some embodiments, the catheter is dimensioned for intravascular delivery and deployment of the implant at a target region through a femoral access site of the patient. Typically, the catheter is between 100-150 cm, preferably about 135 cm. In some embodiments, the catheter handle includes a retractable hub coupled to the outer shaft to allow manual retraction of the outer sheath for deployment of the implant. In some embodiments, the implant is self-expandable, while in other embodiments, the implant is balloon-expandable by expansion of one or more balloons.

In some embodiments, the delivery catheter shaft and/or implant includes one or more visualization markers thereon to facilitate positioning of the implant at the target region within the aortic ach. The one or more visualization markers are disposed on a portion of the implant, such as the frames of the expandable structures, or on the flexible connectors. The catheter can further include one or more markers are disposed on a portion of the catheter shaft and/or the outer sheath, for example the outer sheath includes one or more markers thereon to facilitate retraction of the outer sheath a sufficient distance to deploy part or all of the implant.

In one aspect, the invention pertains to an implant for treating hypertension in a patient. The implant has a collapsed configuration for advancement through a vasculature of the patient and an expanded configuration for engagement of an arterial wall within an aortic arch of the patient. The implant can include one or more expandable structures. In some embodiments, the implant has a total length of at least 60 mm such that, when deployed in the aortic arch, the implant extends along a target zone that extends at least between the brachiocephalic artery takeoff and the left subclavian artery takeoff. In some embodiments, the implant has a non-circular cross-section, when expanded, along its entire length so as to induce wall tension along entire zone of the aortic arch thereby inducing a baroreflex response of baroreceptors in the target zone. The implant has sufficient flexibility to accommodate a curvature of the aortic arch along the elongated zone. In some embodiments, any side openings in the implant are sufficiently large to allow lateral blood flow therethrough to any side branches in the aortic arch and to expose arterial walls to pulsatile blood flow to maintain a long-term response.

In some embodiments, the implant has an elliptical, oval or pill-shaped cross section. In some embodiments, the non-circular cross-section is a regular polygonal shape. In some embodiments, the non-circular cross-section is constant along the entire length of the implant. In some embodiments, the non-circular cross-section is variable (e.g. increasing/decreasing or tapering) along a length of the implant. In some embodiments, a greatest lateral dimension of the implant is greater than 25 mm. In some embodiments, a greatest lateral dimension of the implant is between 40-60 mm. In some embodiments, a total length of the implant is about 60 mm or greater, typically between 70-90 mm. In some embodiments, the implant is self-expandable. In some embodiments, the implant is a woven or braided from one or more wires. In some embodiments, the implant is formed of a plurality of wires (e.g. 12, 24, 36, 48, etc.). Typically, the implant is formed of Nitinol. In some embodiments, the implant is formed from a laser cut tube. In some embodiments, when expanded, the implant design is such that interstitial openings have sufficient area, such as at least 9 mm², typically within a range of 9-30 mm² to allow blood flow therethrough and expose arterial walls to pulsatile blood flow.

In another aspect, the invention pertains to a method for treating hypertension in a patient. Such methods can include steps of: deploying an implant within an aortic arch of the patient, the implant having a collapsed configuration for advancement through a vasculature of the patient and an expanded configuration for engagement of an arterial wall within an aortic arch of the patient; engaging the arterial wall with the implant in the expanded configuration along a target zone that extends at least between the brachiocephalic artery takeoff and the left subclavian artery takeoff, wherein the implant has a non-circular cross-section when expanded thereby inducing wall tension along the target zone in the aortic arch; and exposing a majority of the arterial wall along the target zone to pulsatile blood flow side openings of the implant so as to sustain the baroreflex response induced by the implant long-term.

### BRIEF DESCRIPTION OF THE DRAWINGS

FIG. 1A shows an exemplary implant device deployed along the target region in the aortic arch of a patient, in accordance with some embodiments.

FIG. 1B shows another exemplary implant device deployed along the target region in the aortic arch of a patient, in accordance with some embodiments.

FIG. 1C shows an exemplary implant device deployed along the target region in the aortic arch of a patient, in accordance with some embodiments.

FIG. 1D shows another exemplary implant device deployed along the target region in the aortic arch of a patient, in accordance with some embodiments.

FIG. 1E shows an exemplary implant device deployed along an elongated target region of the aortic arch of a patient, in accordance with some embodiments.

FIG. 2 shows an illustration of a conventional view of the anatomy of the vasculature and baroreceptors and the carotid baroreceptor location targeted by conventional devices.

FIG. 3A shows details of an exemplary type of baroreceptor called PEIZO1.

FIG. 3B shows the central nervous system response to baroreflex stimulation in regulating blood pressure.

FIG. 4 shows the anatomy of the vasculature of the aortic arch.

FIG. 5 shows the embryonic anatomy that later develops into the aortic arch.

FIG. 6 shows the anatomy of the aortic arch illustrating additional details as to a target region, in accordance with aspects of the invention.

FIG. 7 shows histology of the arterial tissue within the targeted region.

FIG. 8A shows a fluorescence staining image from an animal study illustrating the distribution of baroreceptors in the target region.

FIG. 8B shows a fluorescence staining image illustrating the location of baroreceptors within the arterial wall of the aortic lumen.

FIGS. 9A-9B illustrate results from a prior animal study showing heightened sensitivity of the baroreceptors in the aorta as compared to baroreceptors in the carotid.

FIGS. 10A-10D show an exemplary implant having two expandable structures interconnected by flexible connectors, the structures defined by four frames and having a square cross section, in accordance with some embodiments, and FIG. 10E shows an exemplary implant having three expandable structures in accordance with some embodiments.

FIGS. 11A-11B show an alternative embodiment of the implant, where the expandable structures are defined by three frames and having a triangular cross-section.

FIGS. 12A-12B show an alternative embodiment of the implant, where the expandable structures are defined by five frames and having a hexagonal cross-section.

FIGS. 13A-13D show another alternative implant design having three expandable structures, in accordance with some embodiments.

FIGS. 14A-14C show various dimensions of the aorta (A, B, C, D, E) that were tabulated in a patient study to determine appropriate sizing of the implant structure for deployment in the aorta.

FIG. 15A-15D show the mechanism of action by which an appropriately sized structure engages and stretches a substantially round (e.g., 25 mm diameter) aortic arterial wall to achieve sufficient stretch to induce baroreflex, in accordance with embodiments of the invention.

FIG. 16 illustrates a target region of the aorta, which is the entire aortic segment between the LCCA and LSA, in accordance with some embodiments.

FIG. 17 illustrates a delivery catheter configured to deliver and deploy the implant in the aortic arch, in accordance with some embodiments.

FIGS. 18A-18D illustrate delivery of an exemplary implant to the aortic arch along the target region with the delivery catheter, in accordance with some embodiments.

FIGS. 19-20 illustrates methods of treating hypertension with an implant, in accordance with some embodiments.

FIGS. 21A-1 through 21C-3 illustrate alternative designs of expandable structures for aortic arch baroreceptor implants, in accordance with some embodiments.

FIG. 22 illustrates a relationship between compliance of a simulated aortic arch vessel before and after placement of an implant, in accordance with some embodiments.

FIGS. 23A-23B illustrates an exemplary implant utilizing multiple expandable structures, similar to those in FIGS. 21A-1 through 21C-3, interconnected by flexible connectors and deployed in the aortic arch, in accordance with some embodiments.

FIGS. 24A-24F illustrates alternative flexible connectors designs between adjacent expendable structures for use in aortic arch baroreceptor implant, in accordance with some embodiments.

FIG. 25 illustrates another exemplary implant for treating hypertension configured to stretch the arterial walls both circumferentially and axially, in accordance with some embodiments.

FIG. 26 illustrates steps of deploying the implant of FIG. 25 in the aortic arch, in accordance with some embodiments.

FIGS. 27A-27F illustrates a tethered articulated implant and various steps of deployment, in accordance with some embodiments.

FIGS. 28A-28B illustrate an exemplary aortic arch morphology and deployment of a tether articulated implant therein, in accordance with some embodiments.

FIG. 29A illustrates a two-ring implant, in accordance with some embodiments.

FIG. 29B illustrates a three-ring implant, in accordance with some embodiments.

FIGS. 30A-30B illustrate a multi-ring tethered implant, in accordance with some embodiments.

FIGS. 31A-31B illustrate alternative implant structures, in accordance with some embodiments.

FIGS. 32A-32E illustrate various views of an exemplary 8-cell implant structure, in accordance with some embodiments.

FIGS. 33A-33E illustrate various views of an exemplary 4-cell implant structure, in accordance with some embodiments.

FIGS. 34A-34C depict various tether configurations and interfacing of tethers within a retention collar, in accordance with some embodiments.

FIGS. 35A-35B depict tether configurations for 8-cell and 4-cell implant structures, in accordance with some embodiments.

FIGS. 36A-36D depict various views of a retention collar for retaining the implant structure before full deployment, in accordance with some embodiments.

FIG. 37 depicts a constrained implant releasably connected by tethers to a collar of the delivery catheter, in accordance with some embodiments.

FIGS. 38A-38B depicts detail views of tethers of an implant interfaced with the retention collar, in accordance with some embodiments.

FIGS. 39A-39C depicts a setting ring for setting a diameter of the implant structure, in accordance with some embodiments.

FIGS. 40A-40H depicts examples of various implant structures being set to a specified diameter by use of one or more setting rings, in accordance with some embodiments.

FIGS. 41A-42D depicts cross-sectional views of exemplary implants that can be used for baroreflex gauging and hypertension treatment, in accordance with some embodiments.

FIGS. 43A-43B shows an exemplary implant structure that is formed of woven wire and has a non-circular cross-sectional shape to tension the artery in which it is deployed by reshaping the artery, in accordance with some embodiments.

FIG. 43C shows an exemplary implant structure that is laser cut and has a non-circular cross-sectional shape to tension the artery in which it is deployed by reshaping, in accordance with some embodiments.

FIG. 44 illustrates a delivery catheter configured to deliver and deploy the implant in the aortic arch, in accordance with some embodiments.

FIGS. 45A-45D illustrate delivery of an exemplary implant to the aortic arch along the target region with the delivery catheter, in accordance with some embodiments.

FIG. 46 depicts a method of delivering an implant for treatment of hypertension, in accordance with some embodiments.

### DETAILED DESCRIPTION OF THE INVENTION

In one aspect, the invention pertains to an implantable device for treatment of hypertension that is configured for implantation at a target region within the aortic arch to induce a baroreflex response, thereby reducing blood pressure.

### I. Physiological Baroreflex Response

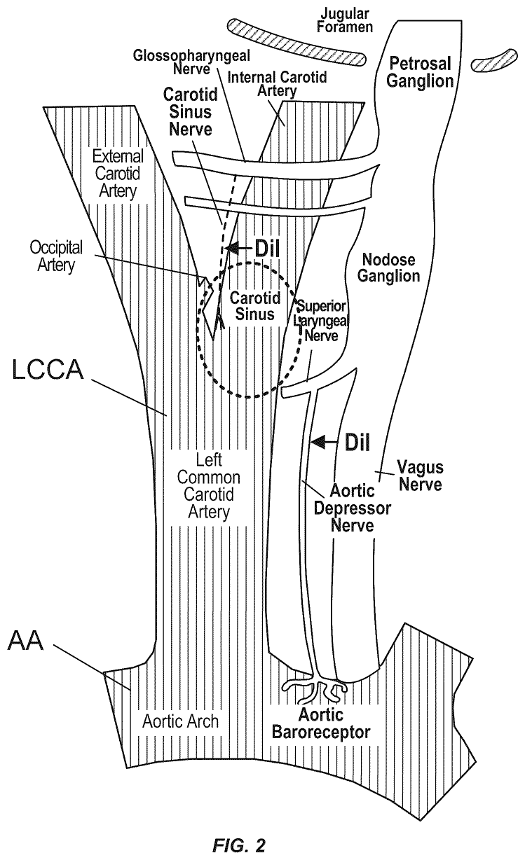

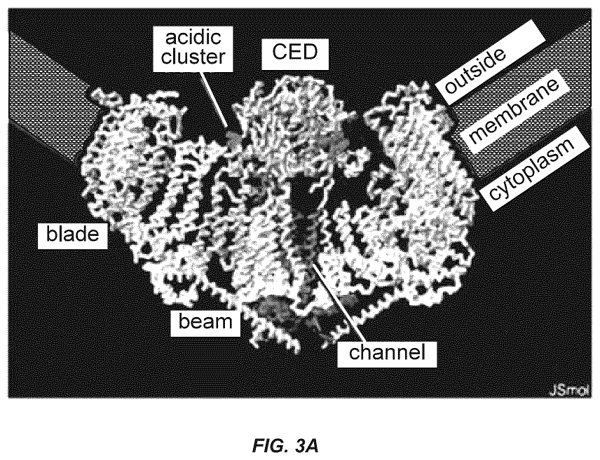

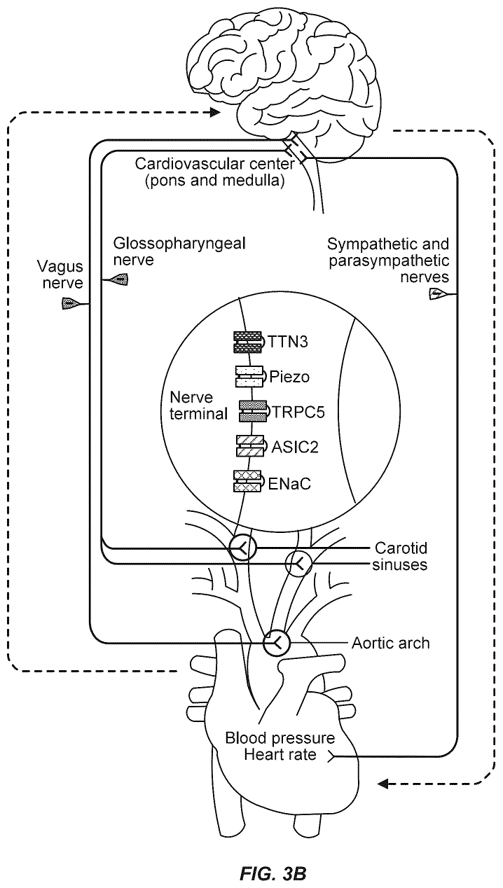

In order to understand the physiological responses of the baroreflex response, it is helpful to understand the interplay between the nervous system within the patient's anatomy. Aspects of the baroreceptor locations with the vasculature, the baroreceptor cells, and the interplay of the baroreceptors with the central nervous system, as shown in FIGS. 2-3B.

Blood pressure sensation occurs at several hotspots within the vascular system. Afferents of the vagus nerve (i.e., cranial nerve 10) and glossopharyngeal nerve (i.e., cranial nerve 9) target the aortic arch and carotid sinus, respectively. Vagal sensory neurons access the aorta through a fine nerve branch termed the aortic depressor nerve, while glossopharyngeal neurons access the carotid sinus through the carotid sinus nerve (see FIG. 2). The aortic depressor and carotid sinus nerves consist of co-fasciculating fibers, including both mechanosensory and chemosensory afferents. The mechanosensory nerve fibers mediate the baroreflex. The terminals of these mechanosensory nerve fibers have specialized nerve endings called baroreceptors that penetrate the artery wall. Baroreceptors are stretch receptors, which do not measure pressure directly, but rather sense stretch of the artery. Blood pressure pulses with each heart beat radially stretch the artery wall, and this arterial distention in turn activates mechanosensitive neurons. Baroreceptor neurons are long aorta-to-brain sensory neurons that transmit inputs directly to the brainstem. Activation of these baroreceptor neurons decreases sympathetic and increases parasympathetic output from the brainstem ultimately lowering blood pressure and heart rate (termed the baroreflex).

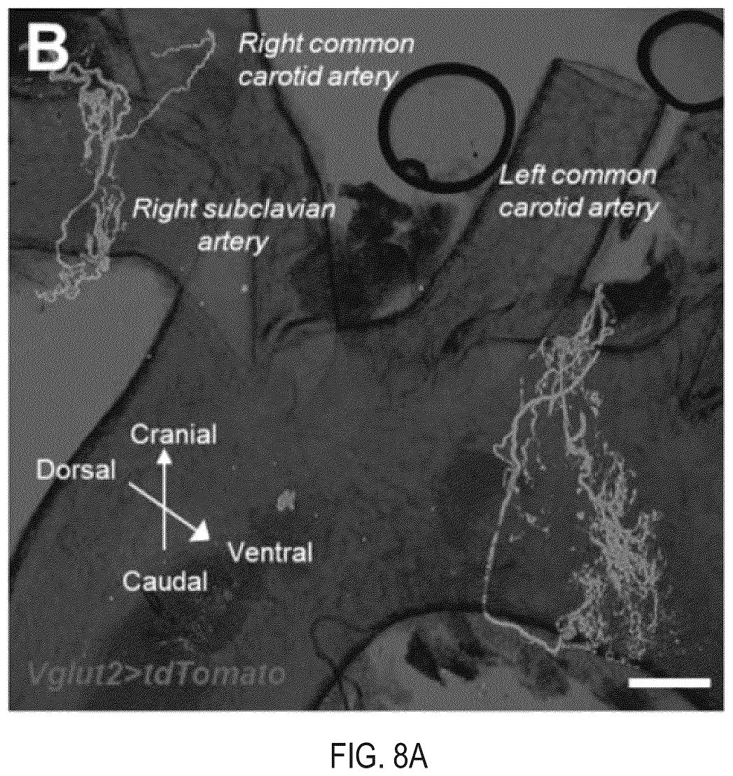

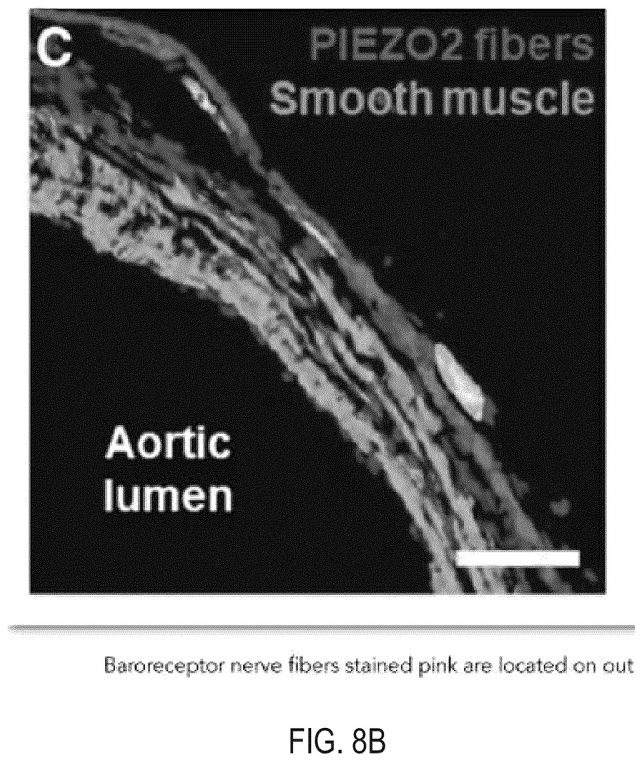

At a molecular level, baroreceptors are actually complex protein structures that form ion channels at the sensory terminals of baroreceptor nerve fibers (see FIG. 3A). Stimulation of the ion channel results in cation influx into the neuron and depolarization with signal transmission along the nerve cell. The baroreceptor nerve terminals are located in the outer wall of the artery between the media and adventitia (see FIG. 8B).

Baroreceptor neurons are long artery-to-brain sensory neurons that transmit inputs directly to the brainstem. The aortic arch baroreceptor nerve signal emanates from the artery wall, travels through the aortic depressor nerve, then via the superior laryngeal nerve to the vagus nerve, and from there, to the brainstem (see FIGS. 2 and 3B). The brainstem modulates this signal reflexively decreasing sympathetic and increasing parasympathetic nerve output to the circulation. Blood pressure and heart rate fall. This cascade of events is termed the baroreflex.

Early animal studies demonstrated that this response was associated with stretching of the arterial walls, which occurs naturally at high blood pressures. Later animal studies demonstrated that the baroreflex response could be transitory or persistent depending upon whether the arterial receptor stimulation was static or pulsatile, respectively. Static stimulus resulted in a systemic blood pressure drop but then normalized a few minutes later, whereas a non-static, pulsatile stimulus (e.g., similar to natural pulsatile blood flow) showed that the systemic blood pressure drop was sustained.

Since the 1960s, modulation of the baroreflex—specifically the carotid baroreflex—has been the primary target of device-based therapy for difficult to control hypertension. The first efforts involved pacemaker-type devices: electrodes placed around the carotid sinus nerve connected via wires to an implantable stimulator. Stimulation of this nerve—which innervates the carotid baroreceptor—circumvents the stimulus within the artery and can lower blood pressure through the carotid baroreflex arc. This technology continues in human clinical trials. A similar type device for the aortic arch baroreceptor or more precisely, the aortic depressor nerve—has been used successfully in a goat experimental model to lower blood pressure. Still, pacemaker-type implants are unlikely to be the device-based solution for resistant hypertension due to their obvious drawbacks—for one, patients would prefer not to have a generator surgically inserted into their chest. To overcome this limitation, Vascular Dynamics developed a stent-like endovascular implant that stretches the carotid artery wall from the inside and amplifies the carotid baroreflex signal. This device has a non-articulated, monomorphic design configured for a straight segment of the proximal internal carotid artery. In early clinical trials, the device was successful at lowering blood pressure in a resistant hypertension population but at the risk of a small number of cerebral transient ischemic attacks (TIA). There are various drawbacks associated with this approach that may contribute to the increased risk of stroke and TIAs.

The present invention seeks to avoid the above-noted drawbacks associated with the conventional approach by providing an endovascular implant that induces an improved baroreflex response within the aortic arch. In an exemplary embodiment, the invention pertains to an implant having one or more expandable structures configured to stretch the arterial walls along a target region of the aorta, in particular the aortic arch. In some embodiments, the invention is configured to engage a target region of the aortic arch, including an inner curvature, opposite the left subclavian artery. This target region can be defined as a cylindrical segment of the aortic arch between the LCCA and the LSA. It is theorized that this portion of the aortic arch is particularly rich in baroreflex receptors and that these receptors have increased sensitivity, as compared to baroreceptors in various other regions, such as the carotid sinus. Since this region of the aortic arch is believed to provide a heightened response, the implant can achieve more consistent, reliable reductions in blood pressure. Further, implantation in this area avoids the drawbacks associated with delivering and implanting at the sensitive area of the carotid sinus, which may increase the risk of TIAs and strokes. Thus, the implant described herein provides a more robust baroreflex response to reduce blood pressure, improves ease of delivery and implantation, and is believed to reduce the risk of adverse events.

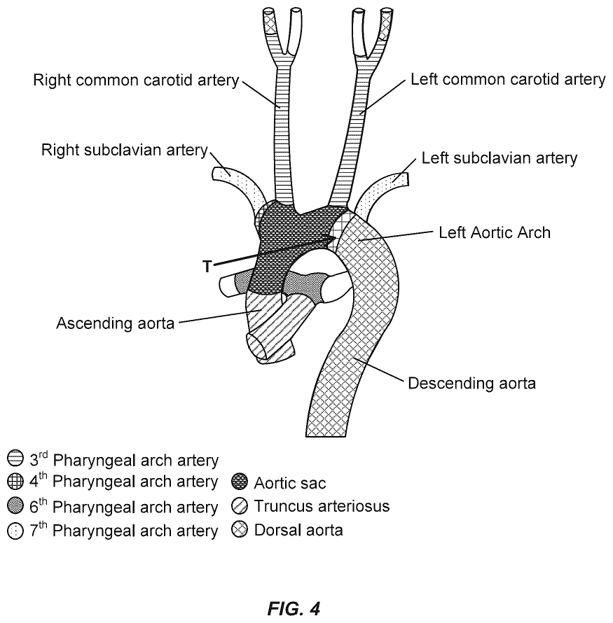

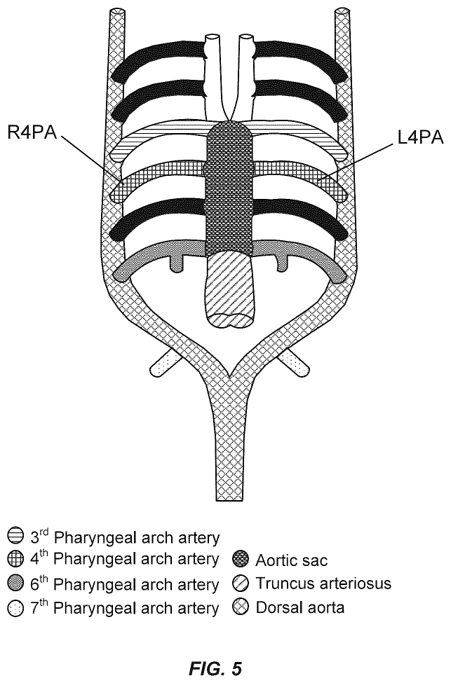

Early animal studies of differing baroreceptor responses have identified anatomic “hotspots” of baroreceptors at various locations in the vasculature, including a narrow cylindrical strip that extends circumferentially around the aortic arch between the takeoffs of the left common carotid and left subclavian arteries (this originates from the left embryonic 4^{th} pharyngeal arch artery as can be seen in FIGS. 4 and 5). This region is shown in the fluorescence staining image of FIG. 8A, which shows the baroreceptors stained in red. This area rich in baroreceptors can be defined as a cylindrical segment of the aortic arch between the LCCA and the LSA, shown as target T in FIG. 4.

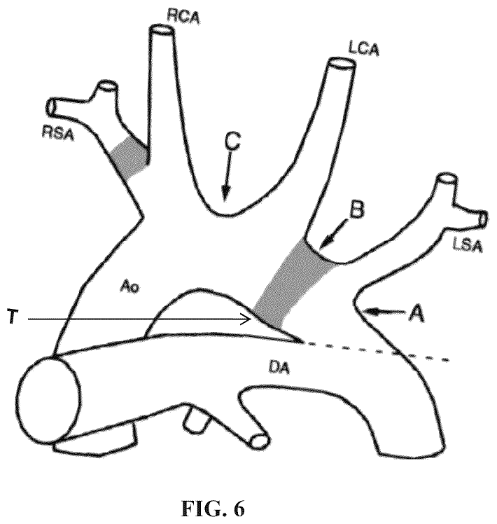

These “hotspots” of baroreceptors originate from the embryonic pharyngeal arch arteries, shown in FIG. 5. The third pharyngeal arch arteries develop into symmetric structures, the right and left carotid arteries. By contrast, the fourth pharyngeal arch artery on the right (R4PA) becomes the proximal right subclavian artery while the left fourth pharyngeal arch artery (L4PA) develops into the relatively narrow cylindrical strip B of the aortic arch noted in FIG. 6. This strip has distinct features as compared to other regions in the aortic arch.

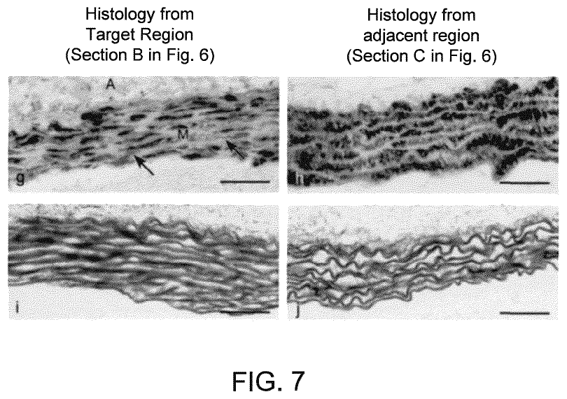

This strip of the aortic arch is histologically distinct from other areas as it has fewer smooth muscle cells and increased elastic lamellae. FIG. 7 shows in the left column, histologic sections through aortic arch segment B, indicating fewer smooth muscle cells and increased elastic lamella as compared to aortic section C in FIG. 6, shown in the right column. It is in this region B that the more commonly known aortic arch baroreceptors are located (as indicated in FIG. 2). However, various animal and human studies have shown there is actually a unique localized distribution of baroreceptors in this region that extends along the band B that wrap the aortic arch, as shown in FIG. 8A. Additionally, the baroreceptors of the aortic lumen are located on an outer layer of the arterial wall within the aorta, as shown in the fluorescence image of FIG. 8B, which shows the baroreceptors nerve fibers stained in pink.

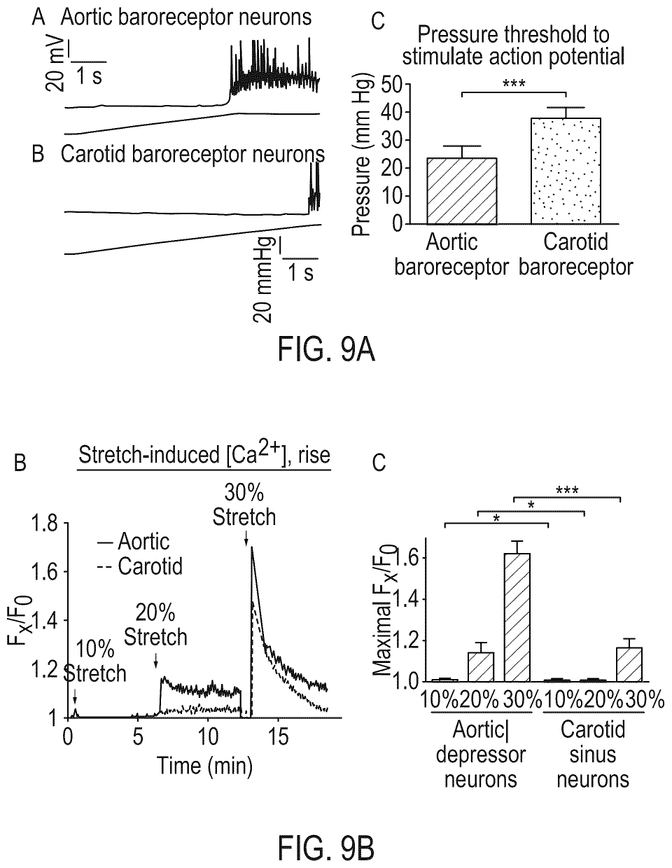

There are very few human reports localizing the aortic arch baroreceptors. One early investigational study indicated baroreceptor nerves wrap the aorta at the original of the left subclavian artery, and the same author later indicated they may extend about the aorta from the brachiocephalic artery to the ligamentum arteriosum. Some early studies have shown that the baroreceptors are found along this region of the aortic arch extending 40% of the circumference, although some baroreceptors may extend outside of this region. Due in part to the histology of this region noted above, it is believed that the baroreceptors behave differently than baroreceptors in other regions, including the carotid artery. At least one animal study demonstrated that the pressure threshold to stimulate action potential was lower in aortic baroreceptors than carotid baroreceptors, see FIG. 9A. The animal study further suggested that a uniaxial stretch of 20% induced a marked increase in cytosolic calcium fluorescence in the aortic arch baroreceptor neurons but not in the carotid baroreceptor neurons, as shown in FIG. 9B. Thus, it is believed that the aortic baroreceptors in the human aorta are more sensitive than baroreceptors in the carotid arteries. Hence, utilizing an implant specifically configured for deployment within the aortic region to stretch the arterial wall in this target region allows for a more consistent pronounced baroreflex response than conventional carotid artery implants.

It is noted that the literature has described the presence of baroreceptors at various locations in the body, including the carotid artery and the aorta, and that some publications describing passive baroreflex implants have mentioned a list of various possible implantations sites in passing, including generally the aorta; however, none have taught any particular target region of the aortic arch. Moreover, none address the unique challenges of implanting in the aortic arch region, challenges which have led the conventional approaches to focus primarily on the carotid artery, which has been commonly accessed to perform certain other procedures, such as placement of carotid stents to address stenosis in this region.

Thus, the present invention seeks not only to provide an improved baroreflex response by implanting at a particular target region in the aortic arch, but also to allow for implantation at this unique site, while improving ease of implantation. Importantly, this claimed implant and approach avoids the drawbacks associated with procedures and implantation within the carotid artery, which can lead to adverse events due to deployment complications in this area.

While there are marked advantages to delivering the implant in the aortic arch, namely the ability to target the aortic arch baroreceptors in the narrow band that wrap the aorta, there are also certain challenges associated with deployment in this area. Since the aortic arch is considerably larger in diameter than the carotid, the implant diameter must be made to correspond to the diameter in the aortic arch in order to sufficiently engage tissues to achieve the requisite reshaping and/or stretching of the arterial walls (e.g., a stretch of 10% or more, 20-50%, or even 20-100%). Further, to prevent flipping or rotation of the implant, the implant should preferably have a length substantially greater than its largest lateral dimension (e.g., diameter), for example about 40 mm or greater. In some embodiments, the length of the entire implant is 70 mm or greater (e.g., about 85 mm).

Another challenge is that the aorta is a main artery within the body such that there is a high volume of blood flow that is carried through the aortic arch as well as into secondary arteries that branch off from the aorta (e.g., BA, LCCA, LSA). Therefore, in order to provide consistent engagement and stretching at the target region long term, the implant must be configured to withstand the forces from the pulsatile blood flow through the aortic arch as well as the lateral forces from blood flow directed into the secondary arteries without dislodging. Further, the implant should be configured to expose a majority of the arterial walls in the target region to pulsatile blood flow, so as to provide the sustained blood pressure drop noted above, rather than a transitory response if the arterial walls were isolated from blood flow. In some embodiments, the implant provides sufficient stretch to induce a baroreflex response that drops blood pressure in hypertensive patients by at least 10 mm Hg, 20 mm Hg, 30 mm Hg, 40 mm Hg, 50 mm Hg, or 60 mm Hg or greater. Yet another challenge is that the implant must allow blood flow freely in a lateral direction into the secondary branch arteries. Thus, the implant itself has been configured with major openings in the expandable structures that both allow exposure of the arterial walls and allow lateral blood flow to feed any adjacent secondary arteries.

### II. Exemplary Delivery Catheter and Implant

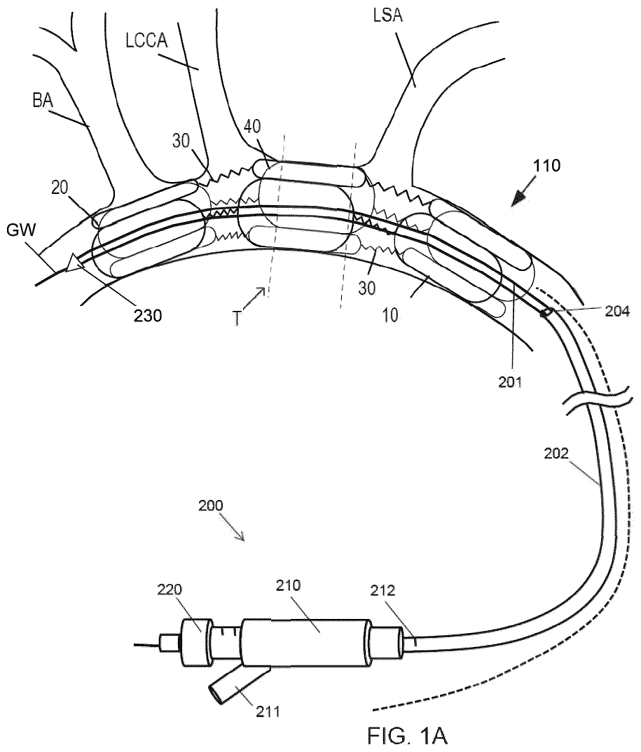

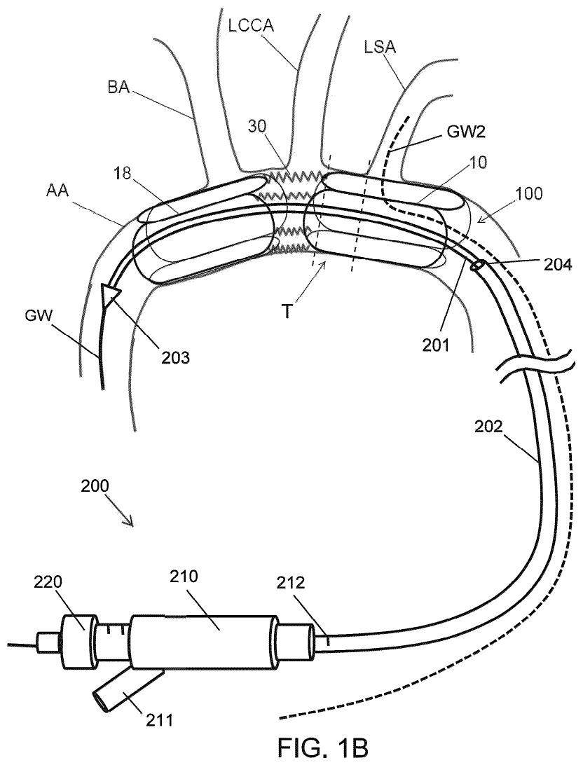

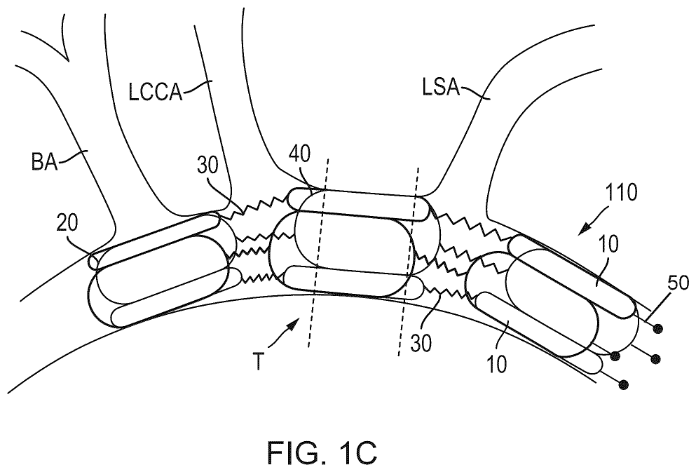

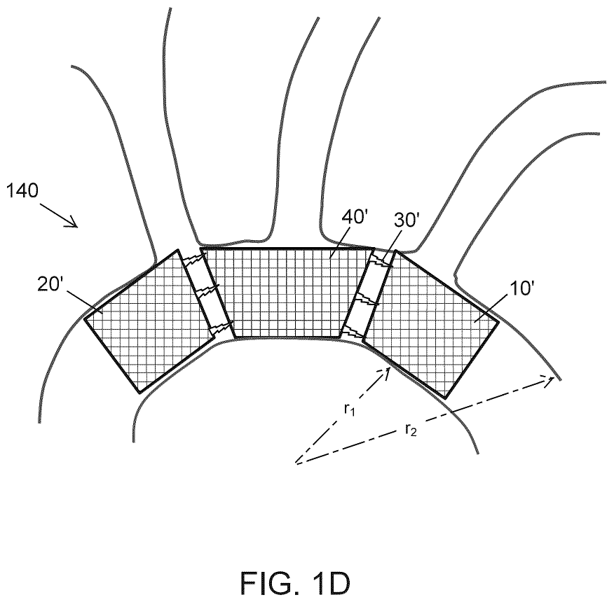

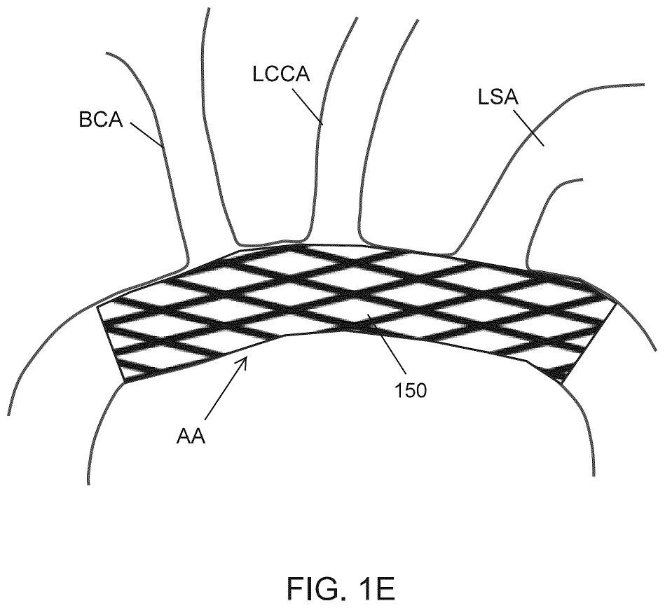

FIG. 1A shows an exemplary implant device 110 for treatment of drug-resistant hypertension that is implanted within the aortic arch AA as delivered by an exemplary delivery catheter 200. In this embodiment, the implant includes three expandable structures 10, 20, 40 interconnected serially by flexible connectors 30, although it is appreciated that more or fewer expandable structures could be used in other embodiments. The delivery catheter system includes an elongated catheter shaft 201 extending between a distal end and proximal end, the shaft having one or more lumens. One lumen can receive a guidewire GW therethrough to facilitate advancement of the catheter over the guidewire GW, previously positioned in the aortic arch. The delivery catheter can further include a tapered distal tip 203 to guide advancement of the catheter over the guidewire GW. The implant 100 is deployed after positioning the distal end portion at the target region within the aortic arch by retraction of an outer sheath 202 constraining the implant in the collapsed configuration during delivery through the vasculature. Retraction of the outer sheath is effected from the catheter handle 210 at the proximal end. The catheter handle can include a hub 220 attached to the proximal end of the outer sheath such that retracting the proximal hub retracts the outer sheath. The outer sheath can include markings thereon to allow the clinician to ensure the sheath is sufficiently retracted to deploy part or all of the implant (e.g., for a self-expandable implant). In other embodiments with a balloon-expandable implant, the delivery catheter can include one or more balloons on the distal portion that can be expanded via the handle. The catheter handle can further include a flush port 211 that is fluidically connected to a lumen extending to the distal end (e.g., a distal opening of the catheter shaft or outer sheath) so that the clinician can flush the aortic arch before, during or after deployment of the implant. The catheter is dimensioned and configured for advancing the implant to the target region T of the aortic arch. The delivery catheter and/or implant can include one or more markers to facilitate precise positioning at the target via visualization techniques. For example, the implant can include one or more markers on one or each of the expandable structures 10, 20, 40 or the flexible connectors 30 between the expandable structures. The catheter can also include one or more markers at various locations, for example, at any of: the distal end of the shaft, the implant location on the shaft, and the distal end of the sheath 204. Optionally, in some embodiments, the catheter system can further include a second guidewire GW2 for placement in a secondary branch, such as the left subclavian artery, to facilitate placement of the implant at the target region relative the LSA. In other embodiments, the implant is positioned relative the anatomy without requiring any additional guidewire.

FIG. 1B shows another exemplary implant device 100 for treatment of drug-resistant hypertension having same or similar components as described in the embodiment of FIG. 1A except the implant includes only two expandable structures that is implanted within the aortic arch AA as delivered by an exemplary delivery catheter 200. The implant is an expandable device inserted into the aortic arch and lowers blood pressure by stretching the aortic arch artery wall from the inside and augmenting the aortic arch baroreflex. Branching from the top of the AA are the secondary branch vessel, the brachiocephalic artery (BA), the left common carotid artery (LCCA) and the left subclavian artery (LSA). While shown deployed with the first expandable structure 10 deployed off-center, it is appreciated that the structure could be centered on the target area T.

As shown, the implant 100 includes two expandable structures 10, 20 interconnected serially by axially expandable connectors 30. The expandable structures are arranged longitudinally along the aorta, which helps anchor and stabilize placement of the implant within the curved aortic arch. Given the relatively large size of the aorta, the high blood flow rate, as well as the curved morphology, anchoring of a single expandable structure in this region can prove challenging. By utilizing two or more structures disposed along differing portions of the aorta, the implant accommodates the curvature and complex geometry of the aorta to help anchor the implant at the target location. Moreover, by relying on engagement of two or more structures along the aorta, the anchoring forces of the implant are distributed over a larger area, thereby minimizing trauma to the arterial walls, which can reduce inflammation and formation of thrombus that can contribute to formation of atherosclerotic plaques.

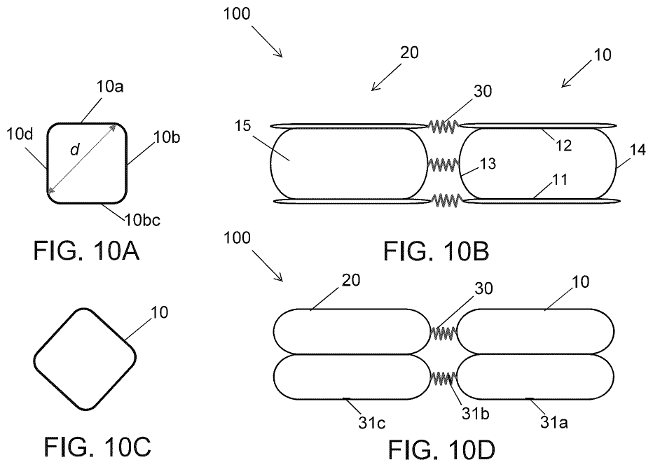

As shown in FIG. 1B, the expandable structures are formed by multiple open wire frames formed by spaced apart struts defining each lateral side. As used herein “frame” can refer to “cells” or similar such structures. The lateral sides of adjacent frames are interconnected along lateral struts so that the frames form a regular polygonal shape, which is axisymmetrical along a longitudinal axis of the expandable structure. The expandable structures have a collapsed configuration for advancement through the vasculature (e.g., within a delivery catheter) and an expanded configuration (as shown) in which the lateral struts engage the arterial walls of the aorta, thereby stretching the arterial walls between each pair of struts in a frame sufficiently to induce the baroreflex response. The flexible connectors 30 are axially expandable (e.g., zig zag connectors) to allow the two expandable structures to extend along differing longitudinal axes so as to accommodate varying degrees of curvature and the complex three-dimensional geometry of the aortic arch. By this configuration, distortion of the aorta or the adjacent great vessels is minimized, and compressive injury to surrounding anatomic structures such as the left recurrent laryngeal nerve is avoided. Additional details regarding the expandable structures can be understood by referring to FIGS. 10A-12B. While these embodiments depict implants defined by interconnected frame, it is appreciated that these aspects are applicable to various other types of expandable structures, for example, expandable structures formed by one or more wires.

FIG. 1C shows another exemplary implant device 110 for treatment of drug-resistant hypertension that is implanted within the aortic arch AA. This embodiment includes three expandable structures 10, 20, 40 that are interconnected serially by multiple flexible connectors 30. This implant is configured so that the middle expandable structure 40 is deployed at the target region T to active the baroreceptors in this region, while the proximal and distal structures 10, 20 act as anchors providing additional stability and also help transition the arterial wall to the middle expandable structure. As shown, the middle expandable structure 40 can have a lateral dimension that is larger than that of the proximal and distal expandable structures. Typically, the middle structure has a lateral dimension that is larger by 1.2-2 times, preferably about 1.3-1.5 times, that of the proximal and distal structures. This configuration allows for further stretching and reshaping of the arterial wall at the target region since the proximal and distal structures stretch the arterial wall to a lesser degree, thereby helping transition the arterial wall to the increased stretch in the target region. This reduces the risk of dissection along the target region from the increased stretch as the proximal and distal structures distribute some of the forces from stretching at the central region. Further, this configuration provides greater stability of the middle structure at the target region. The implant can further include proximally extending tethers 50 to facilitate retraction of a partially deployed implant for removal or repositioning of the implant. Various other aspects of this implant can be similar or the same as those described in FIG. 1A.

FIG. 1D shows another exemplary embodiment for an implant 140, which has asymmetrical structures that better conform to the curvature of the aortic arch and promote more uniform engagement and tensioning of arterial walls along an elongated target region. As used herein “asymmetrical” refers to the expandable structures non-symmetrical from a lateral side view, for example, a trapezoidal in shape with one side being longer than the other as shown in FIG. 1D. As can be seen, the aortic arch has a substantial curvature, the inside curvature having a radius r₁ that is considerably less than a radius r₂ of the outer curvature. In prior embodiments, this curvature led to variable gaps between expandable structures and requires more flexible connectors to accommodate these variations. In this embodiment, the implant 140 includes three asymmetrical expandable structures 10′, 20′, 40′ having a greater length on a side positioned along the outside curvature of the aortic arch and a shorter side positioned along an inside curvature of the aortic arch. These asymmetrical expandable structures allow for a smaller, more uniform gap between structures such that connectors 30′ can be more uniform in length, which allows use of more robust connector designs than prior embodiments. It is appreciated that this implant can include any of the coupling/release features described herein, such as tethers, protrusions, etc., to facilitate controlled delivery and placement at the target region. Since this implant design must be rotationally oriented with the longer side along the outside curvature, the implant and/or catheter can include one or more features to facilitate rotational alignment during deployment, such as one or more markers indicative of a rotational orientation and/or mechanical features to facilitate rotational such as a second guidewire extending through the longer side of the implant and into a lateral side branch extending from the outer curvature side of the aortic arch.

FIG. 1E shows an exemplary implant device 150 for treatment of drug-resistant hypertension that is implanted within the aortic arch AA. In this embodiment, the implant is an expandable device inserted into the aortic arch and lowers blood pressure by tensioning the aortic arch artery walls from the inside with a non-circular (e.g. elliptical, oval) cross-sectional shape, thereby augmenting the aortic arch baroreflex. Branching from the top of the AA are the secondary branch vessels, the brachiocephalic artery (BCA), the left common carotid artery (LCCA) and the left subclavian artery (LSA). As shown, the implant 150 is dimensioned to engage the walls of the aorta sufficiently to reshape the curvature of the walls and is of suitable length to extend along a majority of the aortic arch, at least along a target zone from proximal the brachiocephalic take off to distal of the left subclavian take off. In one aspect, the implant is formed of woven or braided wire to form larger interstitial spaces than in conventional stents, so as to avoid impeding blood flow into lateral arteries and to expose the arterial walls to pulsatile blood flow to maintain a long-term response. In some embodiments, the implant can be laser cut from a tube. In some embodiments, the implant is sized such that the more flattened part of the implant does not touch the arterial walls, at least during a portion of the cardiac cycle, thereby promoting a long-term baroreflex response. It is appreciated that this implant can include any of the coupling/release features described herein, such as tethers, protrusions, etc., to facilitate controlled delivery and placement at the target region. It is appreciated this elongated target region could be engaged by any of the implants and delivery methods described herein.

FIGS. 10A-10D show the exemplary implant device 100 having two laterally expandable structures 10, 20 that are interconnected serially by multiple flexible connectors 30. FIG. 10A shows the cross-sectional view, while FIG. 10B shows a lateral side view. FIGS. 10C-10D show the same views but with the device rotated by 45 degrees along a longitudinal axis. The implant device can further include one or more visualization markers 31 thereon, for example a coating on the flexible connectors 30, to aid in positioning during implantation. In this embodiment, the flexible connectors are axially expandable (e.g., zig-zag connectors) and there are four connectors in total extending between the apex of adjacent crown portions of the first and second expandable structures. FIG. 10E shows exemplary implant device 100′″ having three laterally expandable structures 10, 20, 40 that are interconnected by flexible connectors 30. It is appreciated that some embodiments can further include additional such structures (e.g., 4, 5, 6, etc.) connected in the same or different manner.

In this embodiment, each expandable structure 10, 20 includes four elongated frames (10a/10b/10c/10d) joined along adjacent lateral sides to form a square cross section, as shown in FIG. 10A. Each frame includes at least two linear strut sections 11, 12 defining opposing lateral sides and curved atraumatic crowns 13,14 connecting the proximal and distal ends, respectively. Accordingly, the overall shape of the frame is oblong or pill-shaped. As shown, the atraumatic crowns 13, 14 are gently curved forming an arc of a half-circle or less so that engagement of the proximal or distal ends against tissues does not cause trauma to the arterial wall. The struts and crowns define the overall frame, which leaves a major opening 15 through which the arterial wall is exposed to pulsatile blood flow and which allows lateral blood flow into secondary branch arteries. In some embodiments, the struts of adjacent frames are defined as a single strut, such that a square-cross sectional implant would have only four total struts, one strut on each corner. It is further noted that the entire frame can be formed as a single continuous wire such that the crowns and struts are differing portions of the same wire. In some embodiments, the frames are designed to avoid any sharp corners or angled features of less than 100 degrees, which ensure the proximal and distal ends of the frame remain atraumatic and helps avoid formation of thrombus or plaques within the frame along the major openings through which lateral blood flow is maintained. This design is advantageous as the square cross-section provides sufficient stretch of the arterial walls between the opposite side struts of each frame without overstretching any one portion of the arterial wall, yet still retains normal function and blood flow of the aorta. Although this embodiment includes two expandable structures interconnected by four flexible connectors, the implant could include additional expandable structures connected serially in the same fashion and could include more or fewer flexible connectors.

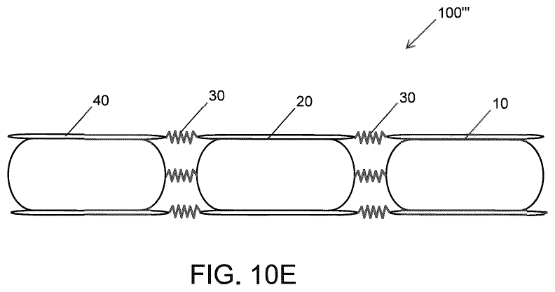

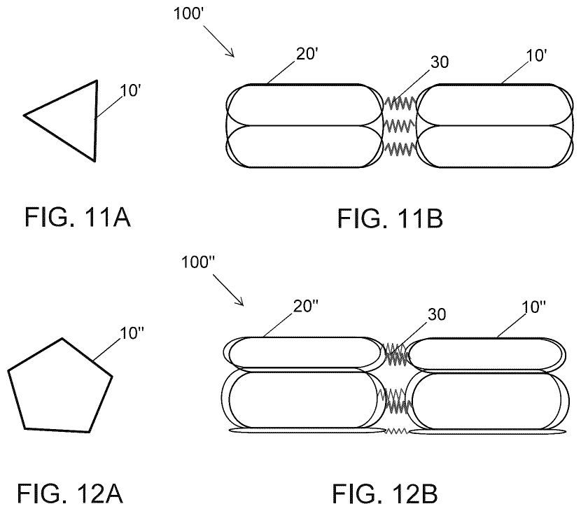

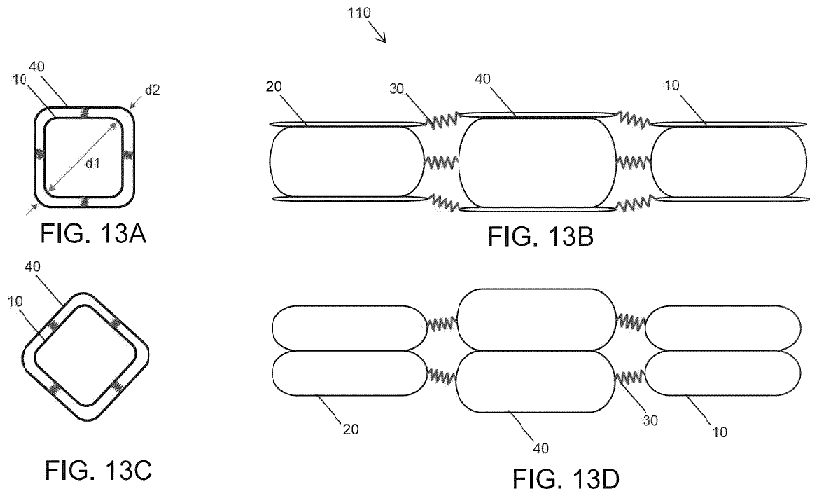

It is understood that these concepts can be utilized in various other shapes/designs, for example triangular or any regular polygonal cross-section, such as those shown in FIGS. 11A-12B. FIGS. 11A-11B show an implant 100′ with first and second expandable structures 10′/20′, each having similar frames as those in FIG. 10A, except each component is formed by three frames such that the cross-section is triangular (e.g., an equilateral triangle). In this embodiment, the largest lateral dimension of the component would be the length of each side of the triangle, which stretches three portions of the arterial wall. FIGS. 12A-12B show yet another implant 100″ having first and second expandable structures 10″/20″, each formed by similar frames as those in FIG. 10A expect each component is formed by five frames to form a hexagon, which stretches five portions of the arterial wall. In this embodiment, the largest lateral dimension would be a distance between an apex and a midpoint of an opposite side. FIGS. 13A-13D show implant 110 (see FIG. 1A) having three expandable structures 10, 20, 40, where the middle expandable structure 40 has a lateral dimension d2 that is larger than the lateral dimension d1 of the proximal and distal expandable structures 10, 20. Dimension d2 can be larger than d1 by 1.2 to 2 times, preferably about 1.3-1.5 times larger. In this embodiment, dimension d2 is 1.3 times larger than d1. As discussed previously, this configuration further improves stability of the implant device in the aortic arch and advantageously allows for further stretching of the target region than would otherwise be safely performed. This is made feasible by utilizing the proximal and distal expandable structures to transition the arterial walls and reduce the risk of dissection or tearing beyond the target region. In other embodiments, the proximal and distal expandable structures could be made larger than the center structure.

In another aspect, the implant is sized specifically for the dimensions of the human aortic arch so as to engage the arterial walls with the lateral struts of the expandable structure so as to anchor the implant within the aortic arch and sufficiently stretch the arterial walls within the target region. In the embodiment shown in FIG. 1, the implant is positioned so that the first expandable structure 10 is positioned opposite the LSA along the target region of the cylindrical band wrapping the aorta, as noted previously. Thus, engagement of a pair of lateral struts in this region stretches the arterial wall and stimulates the highly sensitive baroreceptors in this region. In some embodiments, the implant is sized to achieve a 2:1 implant-to-aorta diameter ratio at the baroreceptor target zone.

### III. Sizing of Implant for Aortic Arch

The baroreceptor amplification device is an endovascular implant designed to amplify the baroreflex response by stimulation of highly sensitive baroreceptors in a precise location within the aorta. This is accomplished by appropriately sizing the implant as described herein to achieve sufficient reshaping and/or stretch (e.g., 20% or more) of the arterial wall within the target region. The implant is dimensioned based on the unique morphology of the aortic arch in humans. In some embodiments, the applicable dimension suitable for such an implant have been determined by a computed tomography angiographic (CTA) study of human aortas. Measurements of the aortic arch CTA were obtained from 50 patients, including both men and women between the ages of 53 and 88. The measurements were tabulated and the means and range were determined per Tables 1 and 2 below.

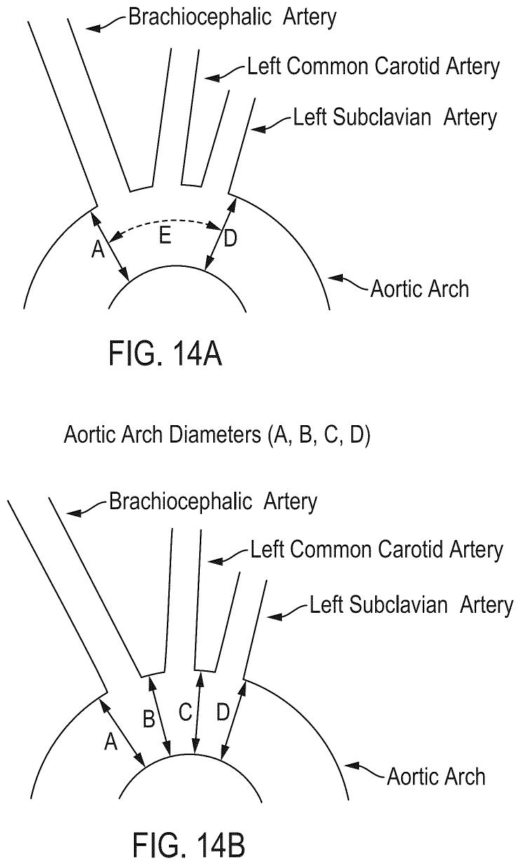

Table 1 shows the mean of various aortic arch measurements, including the aortic arch diameters along regions A, B, C, D (see FIG. 14B) and length E extending between sections A and section D (see FIG. 14A).

TABLE 1
Mean Aortic Arch Measurements
Mean ± Standard Deviation (cm)

| A | B | C | D | E |
|---|---|---|---|---|
| 3.08 ± 0.32 | 2.73 ± 0.29 | 2.44 ± 0.28 | 2.33 ± 0.29 | 3.57 ± 0.64 |

Table 2 shows the range of various aortic arch measurements, including the aortic arch diameters along regions A, B, C, D and length E noted above.

TABLE 2
Ranges of Aortic Arch Measurements
Range (cm)

| A | B | C | D | E |
|---|---|---|---|---|
| 2.54-3.79 | 2.08-3.52 | 1.85-3.03 | 1.87-2.89 | 2.38-5.14 |

In one aspect, the diameter and length dimensions could be considered to display relatively little variation as demonstrated by the small standard deviations and narrow ranges. As shown in FIG. 14C, the aortic arch region has multiple distinct regions where portions of an implant device may potentially be implanted or anchored by one or more portions of the implantable device. Thus, the implant device design should accommodate not only the target region (e.g., typically region E), but may include proximal and distal expandable structures configured to engage the inside diameters along more proximal regions (e.g., region F or G) and more distal regions (e.g., regions A-D) as well. Thus, it is considered that an appropriately sized implant could be made to fit most patients within the above noted ranges. It is noted that arterial walls may be safely stretched up to 50%, potentially up to 100% in healthy patients, such that variability of stretch due to differences in aortic dimensions may be acceptable, so long as the target region is sufficiently stretched (e.g., by at least 20%). In the alternative, it could be considered that these means and ranges of dimension warrant differing sizes of implants. In some embodiments, a set of differently sized implants (e.g., 3-10 different sizes) could be provided and a size could be readily selected based on the particular measurements of the aortic arch of a given patient (see Table 3 below). In another alternative, an implant could be custom-made according to the unique measurement of a patient. The latter two options may be well suited for patients with highly variable morphology or particularly complex geometry of the aortic arch.

In accordance with the above noted means and ranges of the human aorta, the two or more expandable structures can be suitably dimensioned for placement in the aorta. In an exemplary embodiment, each of the expandable structures are between 30 and 60 mm in length, typically about 40 mm in length, and the greatest lateral dimension (e.g., diameter) is between 30 and 55 mm, typically between 30 and 46 mm. These dimensions accommodate a majority of aortas in the average adult human while providing the requisite stretching along the target region to induce the baroreflex response. The expandable structures can be of the same length or of differing lengths and can be the same or differing diameters.

Table 3 below shows a set of differing sizes of implants and associated diameters based on a tabulation of the relevant dimensions of aortic arches of over 50 patients per the CTA study. Component A refers to the more distal expandable structure (20 in FIG. 1), and component B refers to the more proximal expandable structure (10 in FIG. 1) disposed at the target region. As described above, the size of implant can be selected for the unique morphology of a patient based on a CT scan of the patient's aortic arch. It is appreciated that a set of sizes could include any of the sizes noted, or any combination thereof, as well as various additional combinations not listed.

TABLE 3
Size of Implant Configurations (Diameters)

| Size | Component A | Component B |
|---|---|---|
| 1 | 30 mm | 30 mm |
| 2 | 30 mm | 34 mm |
| 3 | 30 mm | 38 mm |
| 4 | 34 mm | 34 mm |
| 5 | 34 mm | 38 mm |
| 6 | 34 mm | 42 mm |
| 7 | 38 mm | 38 mm |
| 8 | 38 mm | 42 mm |
| 9 | 38 mm | 46 mm |
| 10 | 42 mm | 42 mm |
| 11 | 42 mm | 46 mm |
| 12 | 42 mm | 50 mm |

In another aspect, the two or more expandable structures are connected serially by multiple flexible connectors. Preferably, the connectors are axially expandable (e.g., zig-zag design) to optimize conformity to the outer and inner curvatures of the aortic arch. In some embodiments, the connectors are axially expandable by 5-20 mm, typically about 5-10 mm. In some embodiments, the connectors are between 5 mm unexpanded and up to about 10 mm or more fully expanded so that the connectors on the outer curvature of the aortic arch can be expanded while the connectors on the inner curvature of the aortic arch can remain unexpanded, as shown in FIG. 1.

In another aspect, the length of each expandable structure is typically between 30 and 50 mm, preferably about 40 mm, such that the overall length of the entire implant including the flexible connectors is between 65 and 110 mm, typically between 70-90 mm depending on the axial extension of the connectors. These lengths allow the implant to extend a minimum of 10 mm beyond both the lateral aspects of the brachiocephalic artery and the lateral aspect of the left subclavian artery to ensure a safe and stable loading zone for the device. Based upon the CTA study, implant is about 85 mm when the connectors are unexpanded and approximately 10 mm or greater (e.g., 10-20 mm) when the connectors are fully expanded.

Based on previous animal studies, it is believed that human aortic arch baroreceptors need to be stretched a minimum of about 20% to achieve a significant increase in baroreceptor nerve signaling. Consequently, the implant is dimensioned with a greatest lateral dimension or diameter that is a minimum of 20% greater than the natural diameter of the target region (e.g., measurement C from the CT angiographic study). The diameter of the implant should be sufficient to ensure adequate aortic arch wall apposition at the terminal landing zones just beyond the lateral take-offs of the brachiocephalic and left subclavian arteries (e.g., locations A and D in FIG. 13). For sizing purposes, the diameter of the implant is measured as the largest lateral dimension (e.g., for a square cross-section, the diagonal shown in FIG. 10A). Based upon the CTA study, the implant can be sized in various differing diameters, for example, 30, 34, 38, 42, 46, and 50 mm. The implant can be constructed with components A and B of different diameters, for example, as shown in Table 3.

### IV. Mechanism of Action

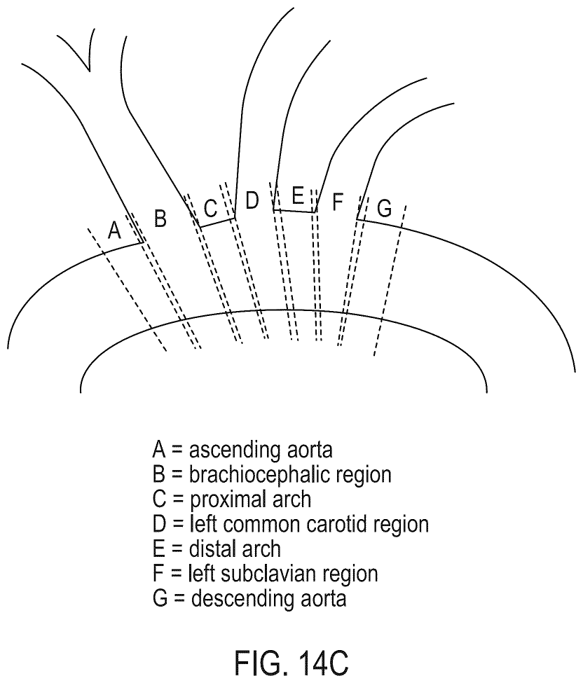

To further understand the sizing of the implant, the mechanism of action by which the implant reduces blood pressure should be understood. It is helpful to consider the aortic arch as a circle in cross-section and to consider the arterial wall in discrete arc lengths, as determined by the figure and the arc length formula shown in FIG. 15A. In the case of an implant having a square cross-section (as in FIG. 10A), the aorta diameter is considered to be a circle divided into equal parts (e.g., four equal parts). If the aortic arch diameter is 25 mm then the radius would be 12.5 mm and each arc length would be 19.6 mm, as shown in FIG. 15B. Following insertion of a 30 mm diameter implant with this same example, the aortic arch radius would be 15 mm and each arc length would be 23.6 mm, but only if the aortic arch remained circular, as show in FIG. 15C.

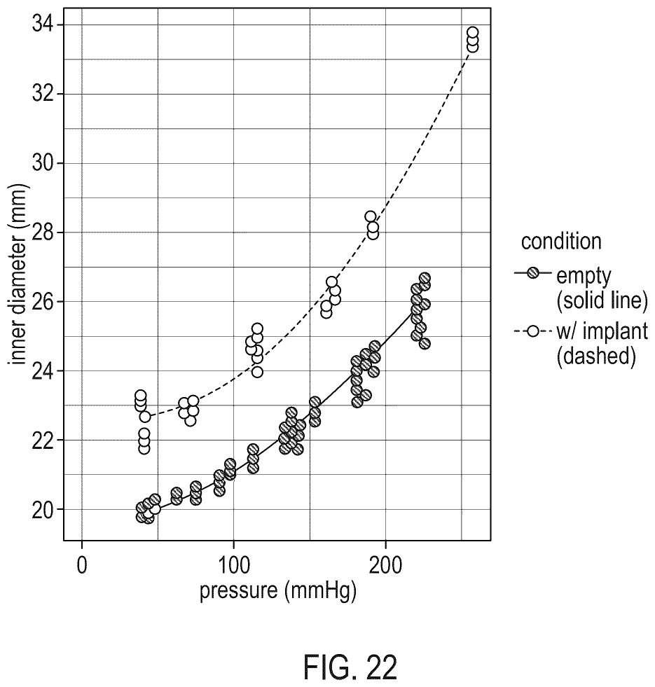

Accordingly, the change in arc length from baseline (FIG. 15B) to post-implant (FIG. 15D) would be an increase of 20% since the radius increases 20% while the other variable stays the same. In other words, each arc of the aorta would be stretched by 20%. However following insertion of the implant, the aortic arch does not remain circular. The radius of curvature of each arc increases, while at the same time the central angle corresponding to that arc decreases (see FIG. 23C). The change of these two variables in opposite directions confounds an exact estimate of the resultant arc length and the extent of the aortic arch stretch, however, this approach provides a reasonable enough estimate of the stretch obtained to appropriately size the implant to achieve at least 20% stretch. It is noted that the analysis above assumes the square cross-section of the implant in FIG. 10A, but this analysis could be modified to account for the implant in FIG. 11A that would divide the cross-section into three equal parts or the implant in FIG. 12A which divides the cross-section into five equal parts.

Thus, by the above approach, the implant can be dimensioned to provide at least a 20% stretch of the target arterial wall. In some embodiments, the implant may be slightly oversized to ensure at least a 20% stretch or to accommodate variations in aorta sizes while still ensuring at least a 20% stretch in all cases. In some embodiments, the implant can be configured to provide additional stretch, for example, 20-30%, 50% stretch, even a 100% stretch may be safely performed in many patients.

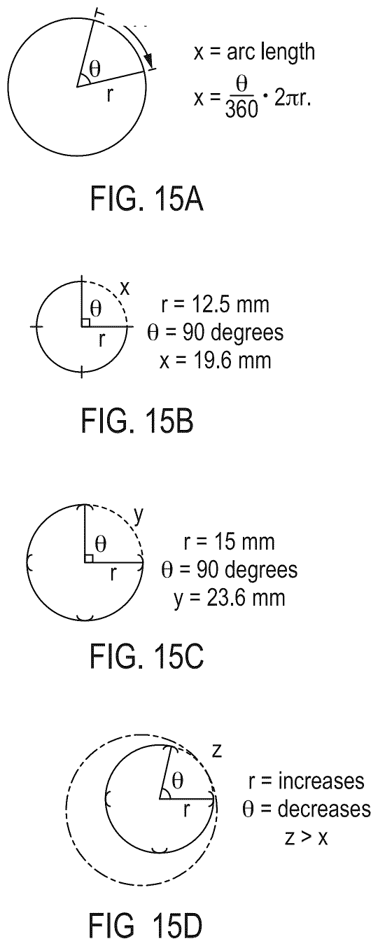

As noted previously, this design allows the device to be deployed and stabilized at a prime anatomic target within the vasculature. Preferably, this target location is within the aortic arch to stretch the aortic arch baroreceptors located along a cylindrical segment of the aortic arch that wraps the aorta between the take-offs of the left common carotid and the left subclavian arteries (including along the inner curvature) from the human aortic arch CT angiographic study. The aortic arch baroreceptors extend along the inner curvature of the aortic arch and extend circumferentially around the arch to the outer curvature or saddle region of the arch, but the greatest concentration of these baroreceptors is located on a segment adjacent the left subclavian artery on the aortic arch that wraps the aorta along diameter C, which is shown as target T in FIG. 16. The implant configuration described herein is specifically configured to target this location but also to stretch adjacent baroreceptors as much as is safe and possible.

### V. Delivery and Placement at Target Region

In yet another aspect, the implant device is especially suited for intravascular delivery and deployment since the implant has a collapsed configuration for advancement through the vasculature and an expanded configuration for engaging the arterial walls, as shown in FIG. 1. In the collapsed configuration, the implant is disposed in a delivery catheter to facilitate intravascular delivery to the target site at the aortic arch and subsequent deployment.

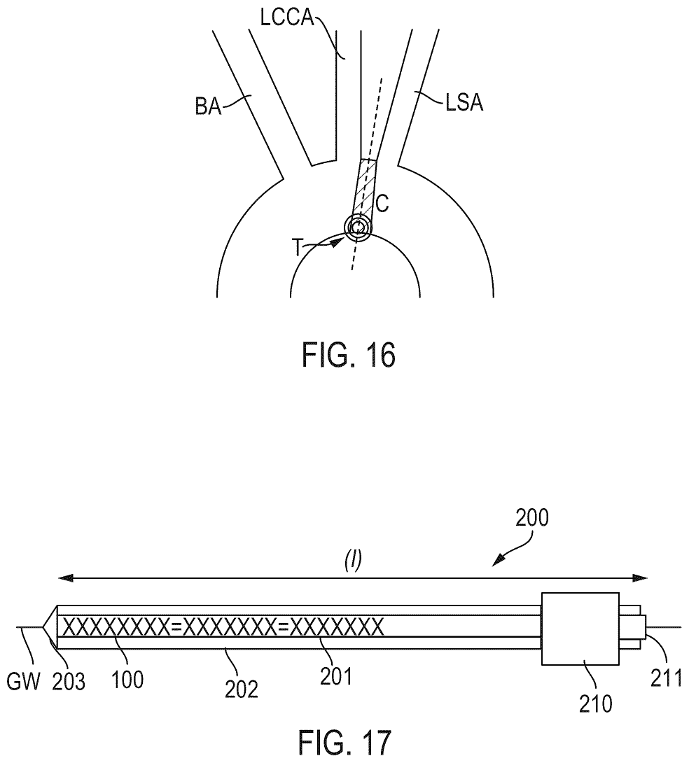

In an exemplary embodiment, the implant is a self-expandable structure that is preloaded into a sheathed delivery catheter, as shown in FIG. 17. As shown, the intravascular delivery catheter is designed to deliver the implant in the collapsed configuration, and to position and deploy the implant at the target location, such as that shown in FIG. 16. The delivery catheter includes an internal guidewire lumen so that it can be advanced along a guidewire GW positioned in the aortic arch. In the embodiment shown, the delivery catheter 200 includes a catheter shaft 201 on which the implant 100 is collapsed, and over which is disposed a retractable sheath 202 that constrains the implant in the collapsed configuration until the implant is positioned at the desired target location, for example by visualization of a marker (e.g., radiopaque or ultrasound marker). The marker can be a coating or marker attached to the connectors, and/or either or both of the expandable structures. In some embodiments, the connectors may be made from a differing material than the frames so that the connectors themselves are distinctly visible through visualization techniques. The delivery catheter can further include a tapered distal tip 203 to guide advancement over the GW and a flush port 211 for flushing before, during, or after delivery. The delivery catheter includes a handle 210 by which the clinician can retract the sheath to deploy the self-expanding implant, which can include a hub 220 for manually retracting sheath 202 to deploy the implant. The sheath and/or shaft can include markings thereon to gauge the distance the sheath is retracted during deployment to facilitate partial incremental deployment or full deployment of the entire implant. Typically, the overall length ( ) of the delivery catheter is between 100-150 cm (e.g., about 135 cm) so as to readily access the aortic arch by insertion of the catheter through the femoral artery.

In some embodiments, the delivery catheter can be configured to deliver the entire implant upon retraction of the sheath, deploying both the first and second expandable structures in rapid succession. The length of the expandable structure is sufficient such that the expandable structure 10 is deployed at the target location despite any minor axial movement upon deployment. Although structure 20 is deployed first, the positioning and deployment is targeting the deployment of structure 10 at the target location. In other embodiments, the delivery catheter can be configured to allow incremental retraction of the sheath by specified distance so as to deliver the expandable structures sequentially, first placing the second, more distal structure, then positioning the first expandable structure precisely at the target location, the axially expandable connectors provide some leeway as to the positioning of the last deployed expandable structure. In still other embodiments, the implant may be balloon expandable and disposed in a collapsed configuration on a balloon of the delivery catheter, the balloon suitably dimensioned for expansion in the aorta to expand and deploy the implant in the target region.

Upon deployment, the implant forms an open lattice with the struts of the frames designed to stretch the aortic arch and stimulate the aortic arch baroreceptors, thereby lowering blood pressure, while the arterial wall is exposed to each aortic pulsation through the major openings of the frames, as shown in the example embodiments in FIGS. 10A-12B.

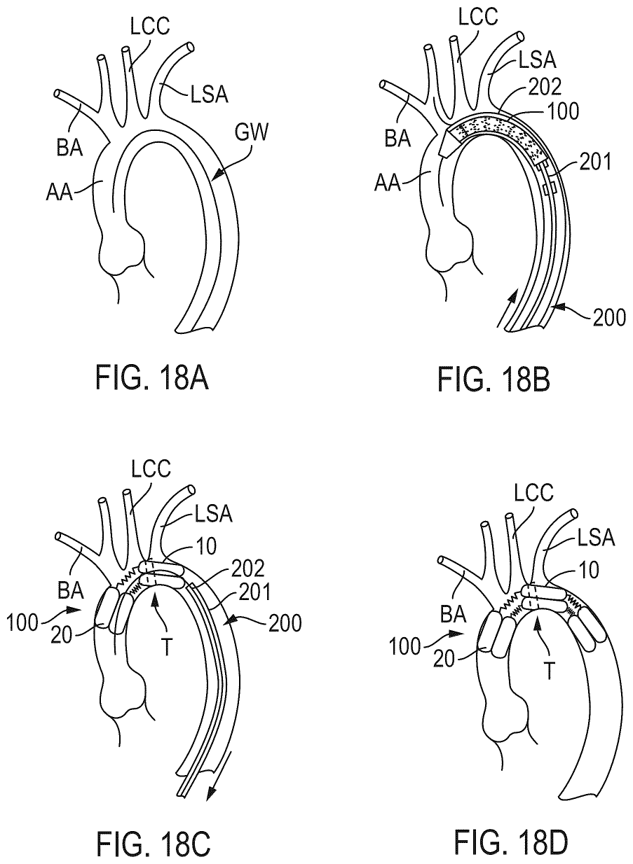

FIGS. 18A-18D illustrate sequential steps of an exemplary method of treating hypertension by deploying the implant device described herein. It is appreciated that this method is exemplary and there may be additional intervening steps or alternative steps in other embodiments.

As shown in FIG. 18A, a guidewire GW is advanced through an entry point (e.g., the femoral artery) and advanced through the vasculature and into the aortic arch. Visualization techniques fluoroscopy can verify placement of the GW in the target region.

As shown in FIG. 18B, the delivery catheter 200 is advanced along the GW, the catheter having an implant 100 disposed in a collapsed configuration on a catheter shaft 201 and constrained within a retractable outer sheath 202.

As shown in FIG. 18C, once the implant is positioned at the desired target location within the aortic arch, the outer sheath 202 is retracted, thereby allowing the self-expandable implant 100 to resilient deploy into its expanded configuration with the two expandable structures 10, 20 engaging the arterial walls.

As shown in FIG. 18D, the guidewire GW and delivery catheter 201 are then withdrawn, leaving the implant anchored at the target location in the aortic arch with at least one expandable structure 100 engaged against and stretching the arterial walls at the target region for long-term reduction in blood pressure.

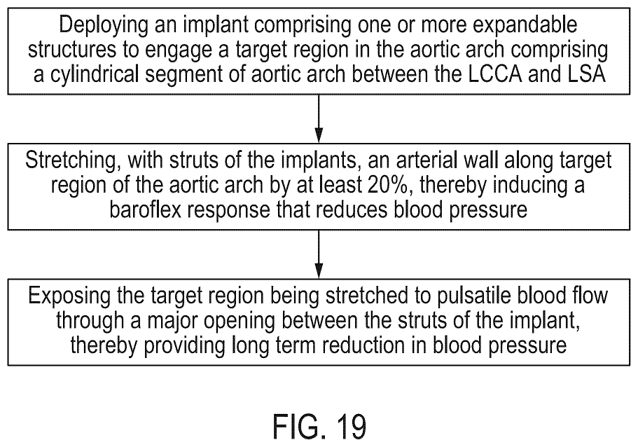

FIG. 19 shows an exemplary method of delivering a hypertension treatment implant device with a delivery catheter. The method includes steps of: advancing a delivery catheter carrying an implant for treating hypertension in a distal portion thereof, the implant being disposed in a collapsed configuration on the delivery catheter to facilitate advancement through the vasculature of the patient; deploying an implant having one or more expandable structures to engage a target region in the aortic arch of a cylindrical segment of aortic arch between the LCCA and LSA; and stretching, with struts of the implants, an arterial wall along target region of the aortic arch by at least 20%, thereby inducing a baroreflex response that reduces blood pressure.

FIG. 20 shows another exemplary method of delivering a hypertension treatment implant device with a delivery catheter. The method includes steps of: advancing a delivery catheter carrying an implant for treating hypertension, the implant having a plurality of expandable structures interconnected by multiple flexible connectors and being disposed in a collapsed configuration on the catheter; positioning the distal portion of the catheter carrying the implant at a target region within the aortic arch, optionally by use of one or more fluoroscopic markers on the catheter and/or implant; deploying the implant to engage the arterial wall so as to stretch the arterial wall along the target region by at least 20%, thereby inducing a baroreflex response of the aortic baroreceptors that reduces blood pressure; and anchoring the implant in the target region long term with the two or more expandable structures, where the flexible connectors axially elongate to accommodate the curvature and complex geometry of the aortic arch, thereby providing long-term fixation.

FIGS. 21A-1 through 21C-3 show alternative designs of expandable structures each defined by one or more wires. Rather than pre-defined frames joined along lateral sides, such as those depicted in FIGS. 10A-13D, each expandable structure can be defined by one or more wires that define a design according to the required dimensions and properties of the implant. In some embodiments, the expandable structure can be formed of a single continuous wire that extends in a meandering, sinusoidal or zig-zag pattern to form a circumferential ring or band of the requisite dimensions to span the target region and exert sufficient outward force when expanded to stretch the arterial wall by the desired amount (e.g., about 20% or more). In some embodiments, the expandable structure is formed by two more wires defined in such a pattern to form a circumferential ring or band of the requisite dimensions and exert the required force. Typically, the wire is Nitinol and is set in a formed diameter that is sufficiently larger than the artery diameter so as to stretch the artery to a target diameter to ensure sufficient stretch of the baroreceptors. The gauge of the wire can be selected to ensure the force requirements are met and ensure longevity of the implant. It is noted that, in many such embodiments, the cross-sectional shape of these expandable structures is substantially circular such that the expandable structure uniformly increases the radius of the vessel wall, while the design still sufficiently exposes the arterial wall to pulsatile forces after deployment.

Three different designs (10A, 10B and 10C) of an expandable structure formed of a wire (e.g., Nitinol wire) are shown in FIGS. 21A-1 through 21C-3. FIGS. 21A-1 through 21C-1 show the structures at the formed diameter. FIGS. 21A-2 through 21C-2 show the structures at the vessel diameter. FIGS. 21A-3 through 21C-3 show the structures in a constrained configuration for delivery to the target location in the aorta. In the embodiments shown, the designs are configured and sized for deployment in the aortic arch. In some embodiments, where the aorta has a vessel diameter in the range of 20-25 mm the expandable structure can be configured such that the formed diameter is between 35-40 mm, typically about 38 mm, which studies have shown exerts sufficient force on simulated vessels of these diameters to effect a suitable stretch of the arterial wall (e.g., typically 20% or more). In these embodiments, the length 12 at the vessel diameter can range between 10-30 mm, typically 10-25 mm. In some embodiments, 10A has a length of about 23 mm, 10B has a length of about 17 mm and 10C has a length of about 14 mm at the vessel diameter. In the constrained configuration, the lengths are slightly longer due to foreshortening effects (e.g., 24 mm, 18 mm and 15 mm, respectively). In some embodiments, the wire diameter is between 0.2 to 1.5 mm, typically between 0.5 mm to 1 mm, more typically about 0.6 mm.

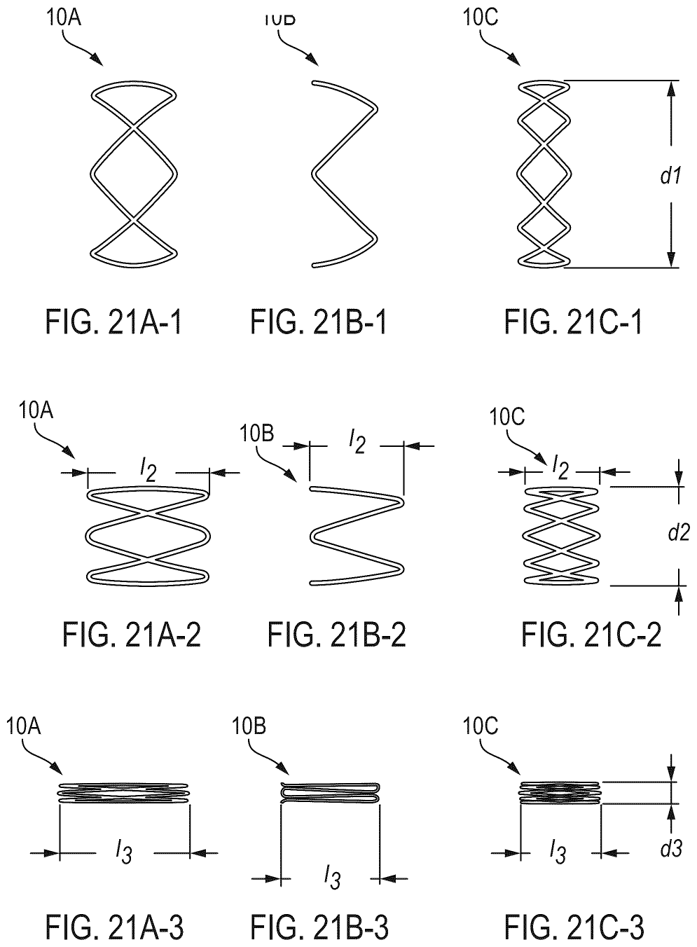

Studies were performed using the wire structure embodiments similar to those in FIGS. 21A-1 through 21C-3 to demonstrate viability of the implants in providing suitable stretch by increasing the diameter of the artery while still allowing sufficient compliance to maintain long term effect. These studies were done in a compliant 0.45 mm resin tube fabricated to mimic the compliance and structure of the arterial wall in the aortic arch. The results of the study are shown in FIG. 22. The compliance of the 0.45 mm resin tube was measured and documented. An exemplary expandable structure was placed into the same 0.45 mm resin tube, and the resulting compliance (dashed line) was plotted together with that of the empty tube (solid line). Both data sets were well fit by linear regression with a second order polynomial.

FIG. 22 demonstrates that the implant ring consistently stretches the resin tube, while preserving compliance. For example, at 50 mmHg pressure, the implant ring increases the diameter from 20 mm to about 23 mm, or 15% stretch. Similarly, at 200 mmHg pressure, the implant ring increases the diameter from 25 mm to about 29 mm, or 16% stretch. The effects of the implant ring can also be interpreted in terms of pressure. For example, at 100 mmHg pressure, the empty tube (representing the native hypertensive vessel) has a diameter of about 21 mm. Placing the implant ring into this tube increases the diameter to about 24 mm, which corresponds to a pressure of 180 mmHg in the empty tube. In this scenario, the outward force of the implant ring is equivalent to +80 mmHg static pressure. In this example, the baroreceptor is stimulated by a stretch of 15%, or an equivalent pressure elevation of +80 mmHg. Therefore, it is hypothesized that the implant ring will trigger a baroreceptor response consistent with such an elevation in pressure, and consequently will decrease systemic pressure.

While it has been shown that acute stimulation of the baroreceptor reflex causes an immediate drop in systemic blood pressure, it has also been shown that a sustained response requires preservation of pulsatility. FIG. 22 also demonstrates that the pulsatility of the empty tube (native vessel analog) is preserved with the implant ring in place (implant ring treated vessel analog). This is evident because slopes of the compliance curves for the empty tube and implant ring tube are substantially parallel. For example, between 100 and 200 mmHg, the empty tube pulses between 21 and 25 mm (19%), while the same tube with a implant ring in place pulses between 24 and 29 mm (21%).

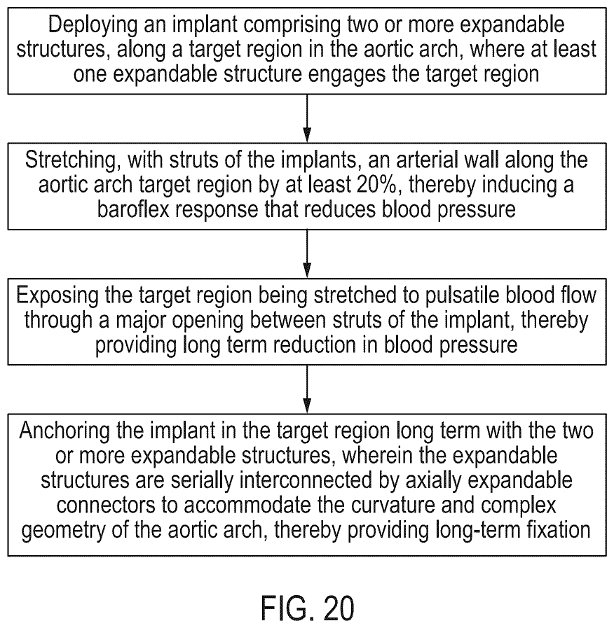

FIGS. 23A-23B illustrate another embodiment 120 that utilizes multiple expandable structures, 10, 20, 40, each of a wire design such as those in FIGS. 21A-21C, and which are interconnected by flexible connectors 30. In this embodiment, D_{V} denotes the nominal vessel diameter. D_{A} is the active segment diameter, which is greater than the D_{V} so as to apply a desired stretch while the expandable structure design preserves pulsatility. D_{D} and D_{P} denote the expanded diameters of the distal and proximal expandable structures, respectively. Each of the D_{D} maximum diameters and D_{P} can be greater than or equal to D_{A} to sufficiently engage the vasculature and prevent migration. The flexible connectors or bridges connect the three expandable structures, each being able to axially elongated or compressed, thereby allowing flexibility to curve match the shape of the arch. For example, the flexible connectors allow more stretching/elongation at the outer radius and less stretching or even compression along the inner radius of the arch.

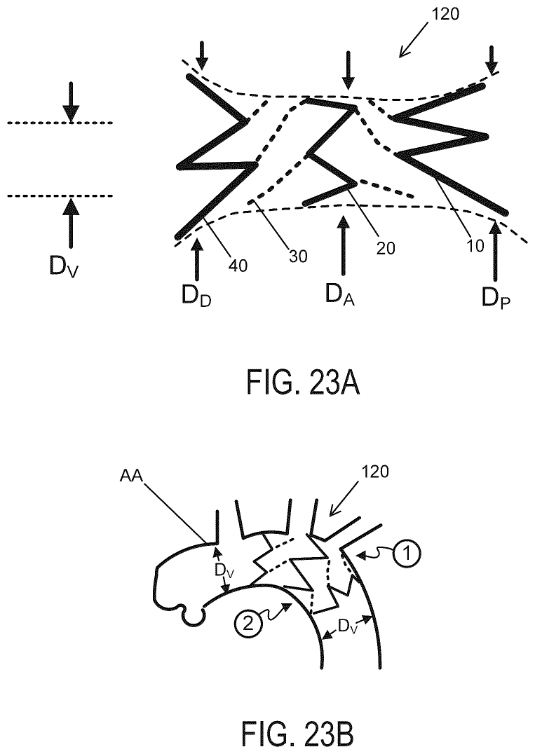

It is appreciated that these wire design expandable structures can also be utilized as the expandable structure of the implants in FIGS. 10-13 and can be similarly dimensioned. For example, the center structure can be dimensioned with a greater diameter than the proximal and distal structures, for example 1.2-1.5 times greater in diameter or largest lateral dimension than the proximal and distal structures. It is further appreciated that various flexible connector designs can be used. FIGS. 24A-24F depict various flexible connector designs that include one or more sinusoidal curves (as in FIG. 24A), V-shaped portions (FIGS. 24B-24D), zig-zag regions (FIG. 24E) or coiled structures (as in FIG. 24F) so as to allow axial expansion between adjacent rings or structures. While particular flexible connectors or bridges are depicted here, it is appreciated that any suitable flexible connectors could be used.

### VI. Alternative Implant Design and Deployment

As described in the previous embodiments, it has been assumed that the implant device utilizes a stimulus of circumferential stretch to activate the aortic baroreceptor nerves. However, it is appreciated that axial stretch may also be relevant and can be utilized to provide additional activation of baroreceptors. In some embodiments, the implant can be configured to stretch the arterial walls in an axial direction in addition to or instead of a lateral direction.

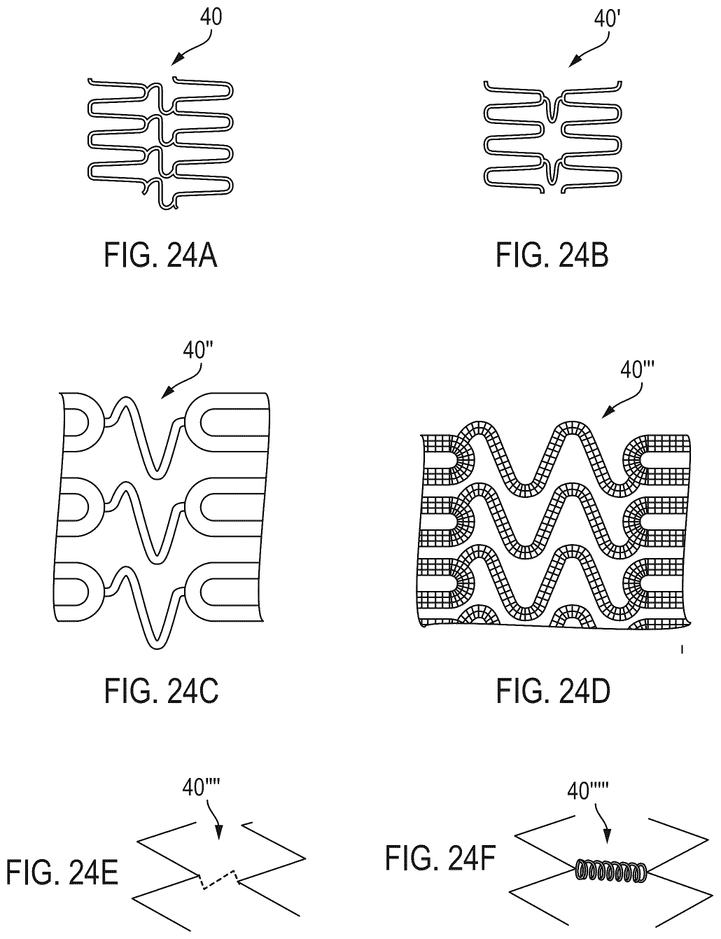

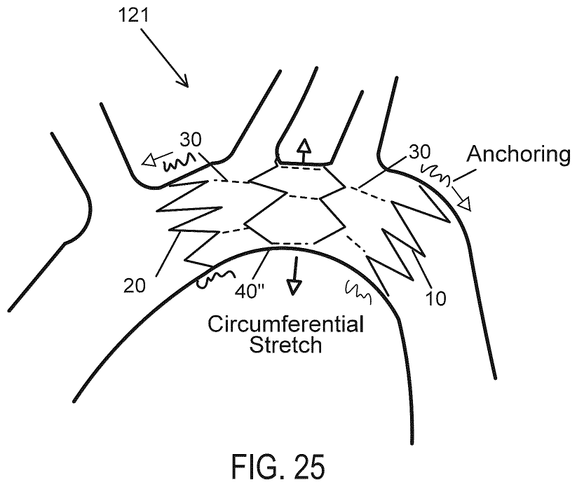

FIGS. 25-26 depict an implant device that is configured to stretch the arterial walls circumferentially/laterally and axially, thereby providing additional activation of the baroreceptors to further enhance the baroreflex response.

As shown in FIG. 25, the implant 12 can include differently configured expandable structures where the middle expandable structure 40 provides the circumferential (e.g., lateral) stretch described throughout the application, and the connectors 30 to the proximal and distal expandable structures 10, 20 are configured to provide axial stretch along the arterial walls. As in some previous embodiments, this design can be a three structure design with proximal and distal anchoring structures and a middle active structure deployed at the target region. In this design, the flexible connectors (i.e., bridges) connecting the proximal and distal structures 10, 20 to the middle active structure 40 are configured as axial springs, which are compressible so as to provide axially directed forces when deployed.

FIG. 26 shows sequential steps of deploying the implant of FIG. 25. In the first step, the distal structure 20 is deployed at a location distal of the target region. In the second step, the middle active structure 40 is deployed at the target region. However, as the middle structure 40 is deployed, the delivery catheter 200 is advanced distally to compress the axial springs between the distal structure 20 and middle structure 40, which axially loads the flexible connectors so as to provide an axially directed stretching force to the arterial wall between the distal structure and the middle structure 40. In the third step, as the proximal structure 10 is deployed the connectors 30 between the middle structure 40 and the proximal structure 10 are compressed by pushing the delivery catheter again during deployment, such that the compressed bridge connectors provide an additional axially directed stretching force from the middle structure 40 to the proximal structure 10. Accordingly, this approach can provide not only circumferential stretch at the target region by the deployed middle active structure, but also provides axial stretch from the target region in both proximal and distal directions, thereby providing enhanced baroreceptor activation or potentially equivalent baroreceptor activation at a reduced implant diameter. While a particular design is described here, it is appreciated that various other designs of the connectors and/or the proximal and distal expandable structures could provide axially directed stretching forces to provide enhance baroreceptor activation.

Accordingly, the implant devices and associated methods described herein address the unmet clinical need to treat patients with severe hypertension unresponsive to multiple pharmacologic agents. Existing conventional treatment and therapies (e.g., renal denervation, carotid artery devices) have had minimal or limited impact on this population due to their limited blood pressure lowering effect or risk of adverse events, respectively. The presently described implant is designed to fulfill this unmet clinical need based on historical and animal studies and identifying the unique anatomy and physiology of the aortic arch baroreceptors and the CT angiographic study outlined above. The implants described herein allow for sufficient stretching of a particular target region of the aortic arch which triggers highly sensitive baroreceptors, thereby consistently and reliably lowering blood pressure in the patient, while avoiding the adverse risks and drawbacks associated with conventional approaches targeting other vasculature, such as the carotid artery.

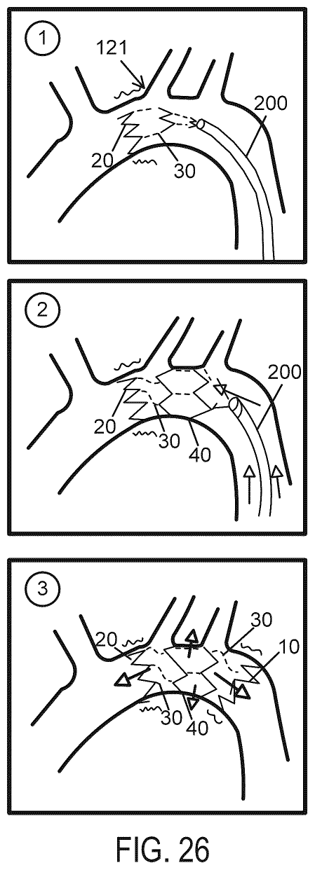

FIGS. 27A-27E illustrates a tethered articulated implant 130 and various steps of deployment, in accordance with some embodiments. FIG. 27A shows an implant structure 130 defined as three expandable rings 10, 20, 40 joined by helically oriented bridges 31 in a reduced diameter D1 for delivery to the target location, as described previously. Each ring can have a sinusoidal or zig-zag shape having peaks and valleys. In some embodiments, the design is formed from one or more wires. In some embodiments, the design is laser cut from a tube (e.g., Nitinol tube). The design further can further include tethers 50 that extend proximally to engage a tool or delivery catheter. In this embodiment, each tether is attached to a proximal apex of a peak of the proximal-most structure 10 and terminates in a proximal connector that is configured to releasably couple with the delivery catheter or tool. FIG. 27B shows the implant structure having been deployed to an expanded diameter D2. In some embodiments, the implant structure can be configured for uniform expansion to a diameter that is 2-4 mm greater than the nominal diameter of the aortic arch. As shown in FIG. 27C, the bridges are configured such that the outer rings (i.e., proximal and distal expandable structures 10, 20) are twisted relative to the center ring, increasing the circumferential orientation of the bridges. This configuration improves flexion hinge properties, and also reduces the total length of the implant. In some embodiments, the bridges hinge laterally so that a distance between expandable structures (e.g., rings) is variable (e.g., shorter between structures along an outer side of the aortic arch and longer between structures along the inner side of the aortic arch) so as to better accommodate the curvature of the aortic arch. This contrasts with conventional designs having longitudinal struts that maintain a spaced-apart, set distance between adjacent structures, which generally is less able to accommodate the tight curvature of the arch. It is appreciated that various other bridge orientations could be used as well, and that the implant structure could be configured for greater diameters or variable diameters as well. FIG. 27D shows an embodiment in which the center expandable structure 40 (i.e., center ring) has a greater deployed diameter than the proximal and distal expandable structures (i.e., outer rings). By this approach, the center ring is configured with a greater diameter to sufficiently stretch the target region to activate baroreceptors in the target region, while the outer rings have a reduced diameter and are configured merely to engage the arterial walls sufficiently to anchor the center ring at the target region and provide a transition to the stretched target region to inhibit arterial damage. Typically, the outer rings are disposed in locations outside the target region rich in baroreceptors. In this embodiment, the center ring is expanded to a greater diameter D3, increasing the outward force delivered to the arch at the baroreceptor target location. In some embodiments, it is appreciated that beyond the stretching of the arterial wall by the expandable structures, the tissue properties are not otherwise altered. FIG. 27E illustrates the tethered articulated implant 130 deployed in the curved aortic arch AA from the delivery catheter 200, the proximal connectors of the tethers still being coupled with a distal portion of the delivery catheter, which can allow for retrieval and repositioning of the implant. The circumferentially oriented hinges separate at the outer radius, and compress at the inner radius, allowing the entire implant to conform to the curvature of the aortic arch. In this embodiment, the tether includes sinusoidal portions that stretch to allow angular transition between the catheter tip and proximal ring of the implant. As shown in FIG. 27F, the sinusoidal tethers can extend to differing lengths, L1, L2, L3, thereby accommodating the curve of the arch. It is appreciated that in other embodiments, shorter tethers can be used without any sinusoidal or stretching portions such that the proximal-most structure remains only partly deployed.

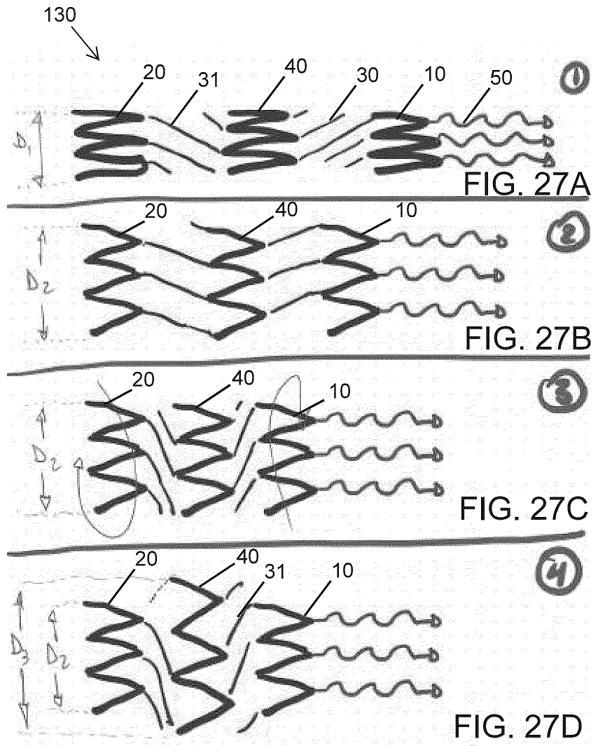

FIGS. 28A-28B illustrate an exemplary aortic arch AA and deployment of a tethered articulated implant 130 therein, in accordance with some embodiments. FIG. 28A shows an exemplary curvature of the aortic arch which has a relatively tight curvature. FIG. 28B shows the implant 130 having helically oriented bridges and proximally extending tethers 50, which allow for controlled positioning, retrieval, repositioning, and removal of the implant by a tool or the delivery catheter 200 if necessary. Although shorter, straight tethers could be used, in this embodiment, the tethers are axially flexible (e.g., sinusoidal, zig-zag portions), which allows the rings to fully expand, oppose the arch, and conform to arch curvature. In this example, the outer tether is extended to a length of about 27 mm, while the inner is extended to a length of about 17 mm. In this embodiment, each tether can be of a sinusoidal design that can stretch from about 20-30 mm when fully extended.

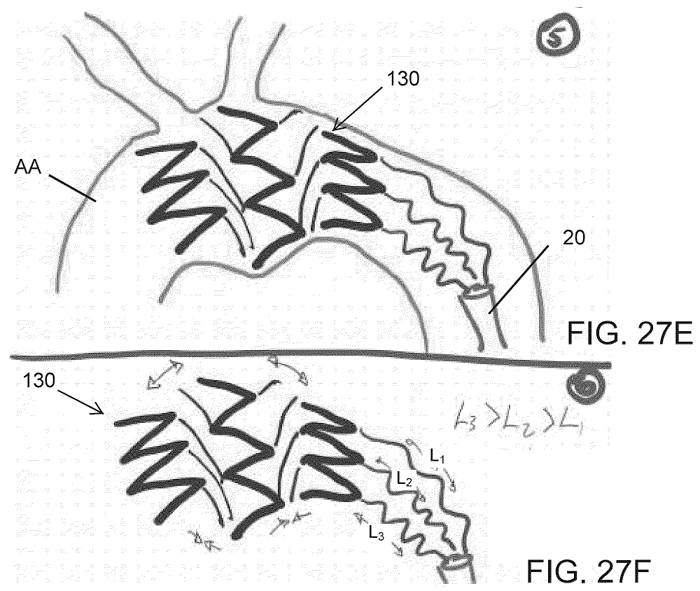

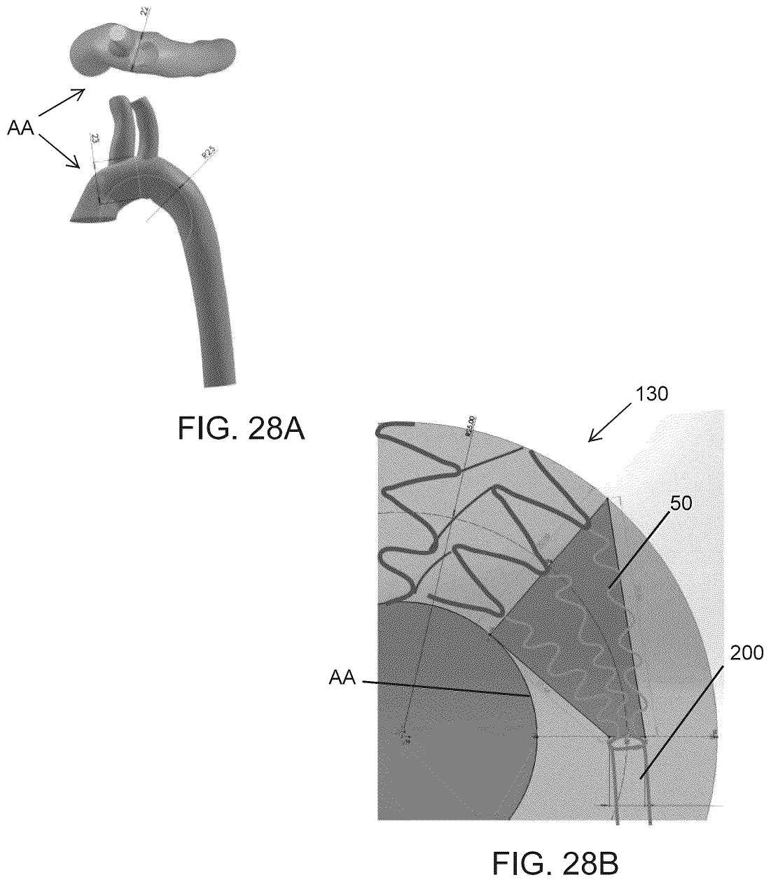

FIG. 29A illustrates a two-ring implant, in accordance with some embodiments. The prototype design includes two expandable structures 10, 20 (i.e., 2 rings) that are joined by helically oriented bridges 31. The active helical bridges provide hinging and stretching movement between the expandable structures. This design can be used to form an implant of any suitable material, including an expandable structure that is laser cut from a metal tube (e.g., Nitinol tube).

FIG. 29B illustrates a three-ring implant, in accordance with some embodiments. The prototype design includes three expandable structures 10, 20, 40 (i.e., 3 rings) that are joined by helically oriented bridge structures 31. The active helical bridges provide hinging and stretching movement between the expandable structures, thereby accommodating the curvature of the aortic arch. This design can be used to form an implant of any suitable material, including an expandable structure that is laser cut from a metal tube (e.g., Nitinol tube).

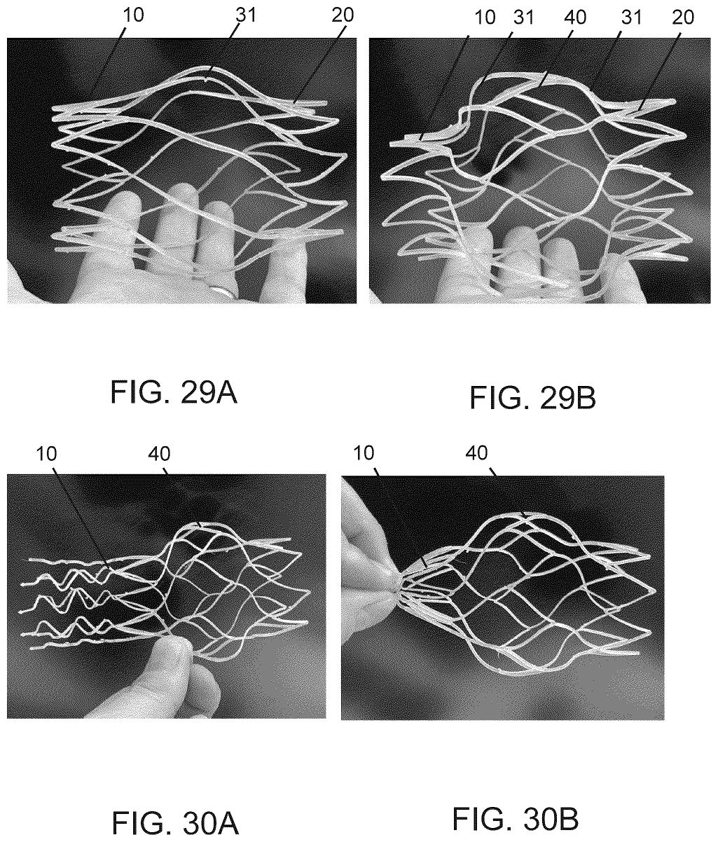

FIG. 30A-30B illustrate a multi-ring tethered articulated implant, in accordance with some embodiments. The prototype design includes three expandable structures 10, 20, 40 (i.e., 3 rings) that are joined by helically oriented bridge structures 31 and extendable tethers 50 extending from the proximal most ring. The active helical bridges provide hinging and stretching movement between the expandable structures, accommodating the curvature of the aortic arch and are of sufficient length such that the center ring can be fully expanded while the tethers are still attached to the delivery catheter, as demonstrated by FIG. 30B, thereby allowing the center ring to be used as a gauge of blood pressure response. This design can be used to form an implant of any suitable material, including an expandable structure that is laser cut from a metal tube (e.g., Nitinol tube). As in previous embodiments, the center ring 40 can be defined so as to have a greater diameter than the outer rings. Varying the diameter of the center active ring varies the amount of “oversizing” applied by the implant to the arch. In some embodiments, it is recommended to shape the implant to have a center active ring at a diameter between 25 mm and 40 mm, typically between 25 mm and 30 mm.

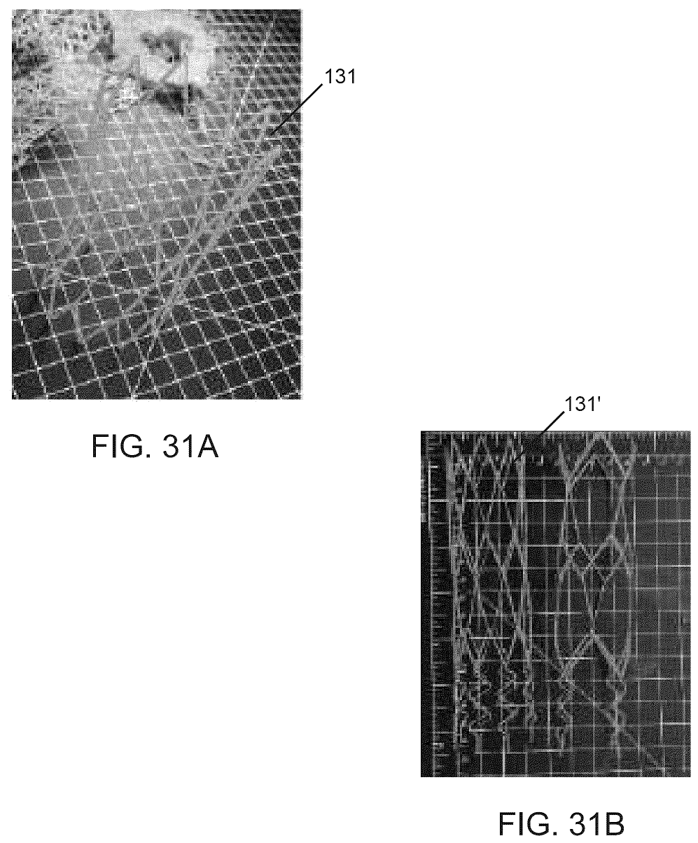

FIGS. 31A-31B illustrate alternative implant structures 131, 131′, in accordance with some embodiments. While particular diamond-shaped patterns are used, it is appreciated that the expandable structures could utilize various other expandable shapes and patterns as well.

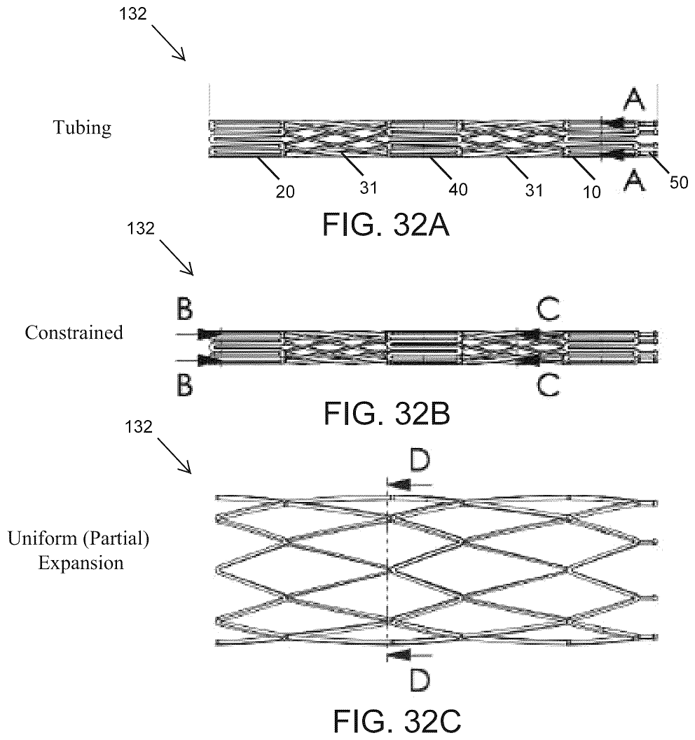

FIGS. 32A-32E illustrate various views of an exemplary implant 132 having three expandable structures 10, 20, 40 (i.e., 3 rings) joined by helically oriented bridges 31 and having short tethers 50 extending proximally, as shown in FIG. 32A, thereby defining an 8-cell structure. The implant can be laser cut from a Nitinol tube (e.g., 5.0 mm diameter tube), or formed by any suitable means. FIG. 32B shows the implant 132 in a constrained configuration. FIG. 32C shows uniform partial expansion of the implant 132 (when fully expanded the center ring can have a greater diameter than the outer rings). FIG. 32D shows various cross-sectional views of the implant. FIG. 32E shows an unrolled view of the implant 132, illustrating the three expandable rings and the 8 helically oriented bridges that define the 8-cell structure noted above. It is appreciated that differing widths of the struts and ring portions of the design can be used to provide differing strengths to provide a desired force on the vessel wall. The expanded diameter corresponds to an equivalent stretch and outward pressure exerted by the implant. For example, in a high strength 8-cell design, the expanded diameters 20 mm, 25, mm and 30 mm may correspond to a stretch of 4, 13 and 23% respectively, and an outward pressure of 100, 356 and 628 mm Hg, respectively. In a moderate strength 8-cell design, the expanded diameters 20 mm, 25, mm and 30 mm may correspond to a stretch of 2, 7 and 12% respectively, and an outward pressure of 39, 140 and 247 mm Hg, respectively.

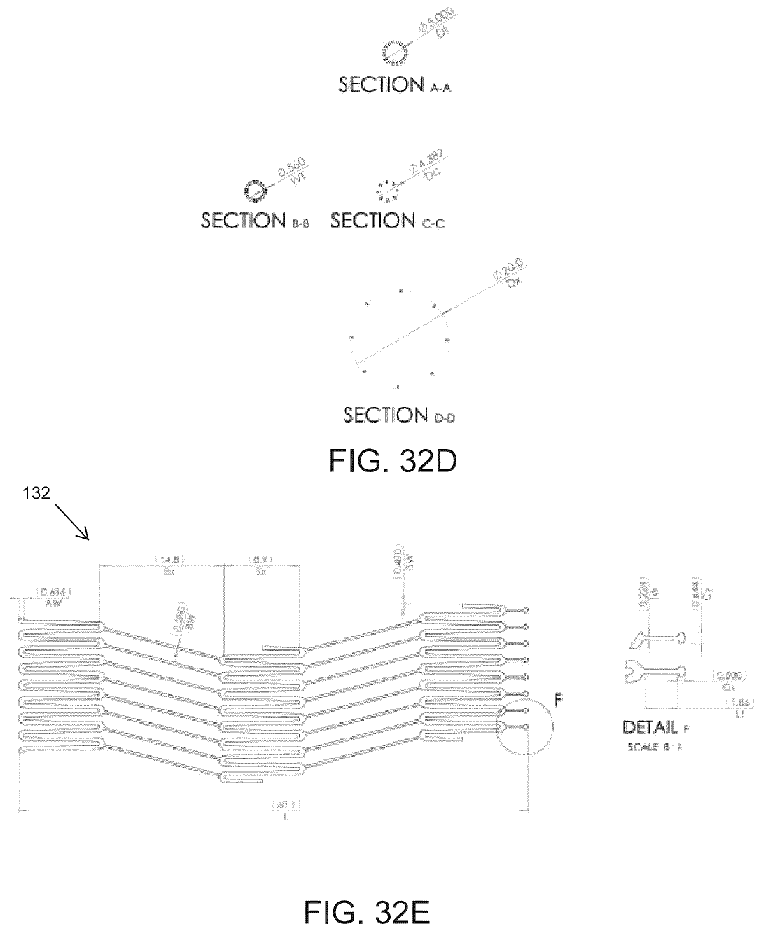

FIGS. 33A-33E illustrate various views of another exemplary implant 133 having three expandable structures 10, 20, 40 (i.e., 3 rings) joined by helically oriented bridges 30 and having short tethers 50 extending proximally, as shown in FIG. 33A, thereby defining a 4-cell structure. The implant 133 can be laser cut from a Nitinol tube (e.g., 5.0 mm diameter tube), or formed by any suitable means. FIG. 33B shows the implant 133 in a constrained configuration (e.g., constrained to 4 mm). FIG. 33C shows uniform partial expansion of the implant 133 (when fully expanded the center ring can have a greater diameter than the outer rings). FIG. 33D shows various cross-sectional views of the implant. FIG. 33E shows an unrolled view of the implant 133, illustrating the three expandable rings and the 4 helically oriented bridges that define the 4-cell structure noted above. It is appreciated that differing widths of the struts and rings of the design can be used to provide differing strengths to provide a desired force on the vessel wall. The expanded diameter of a higher strength corresponds to an equivalent stretch and outward pressure exerted by the implant. For example, in a high strength 4-cell design, the expanded diameters 20 mm, 25, mm and 30 mm may correspond to a stretch of 1, 5 and 9%, respectively, and an outward pressure of 25, 105 and 190 mm Hg, respectively. In a moderate strength 4-cell design, the expanded diameters 20 mm, 25, mm and 30 mm may correspond to a stretch of 1, 2 and 4%, respectively, and an outward pressure of 11, 41 and 74 mm Hg, respectively.

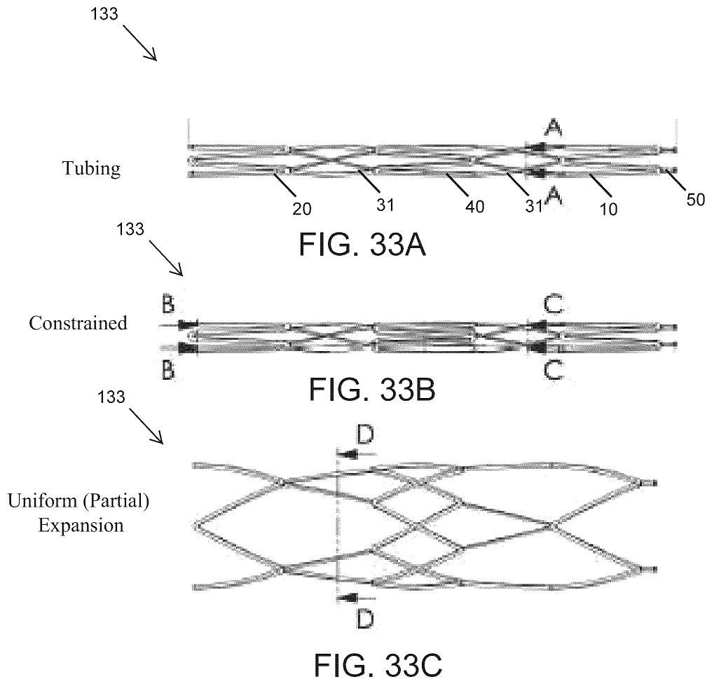

In another aspect, the delivery system includes a lock element that releasably couples with a connector of the implant to allow the implant to be axially adjusted for precise placement at the target location. Preferably, the lock element is included inside the distal portion of the delivery catheter and releasably couples with the implant such that axially advancing the lock element near the distal end partially deploys the implant, while the connectors remain coupled to the lock element. In some embodiments, the connectors are disposed at proximal ends of tethers extending proximally from the implant. Preferably, the implant and lock element are configured such that the center ring of the implant can be fully deployed while the tether remains secured to the lock element. This advantageously allows the clinician to fully deploy the center ring at the target region to assess a baroreflex response before fully deploying the implant. If the response is satisfactory, the outer sheath of the delivery catheter can be further retracted to release the implant from the lock element for full deployment. If the response is not satisfactory, the implant can be drawn back into the delivery catheter by retracting the lock element. The delivery catheter can then be adjusted to a different location for repositioning of the implant, and the baroreflex response assessed at the new location. Alternatively, the lock feature can be axially adjusted to reposition the partially deployed implant without retracting the implant fully back into the delivery sheath. In some embodiments, the lock element is a collar that is coupled to a distal end of a delivery shaft that advances the implant through the delivery sheath. In some embodiments, the collar can include a groove or recess to receive one or more inwardly projecting proximal connector portions of the tethers. In other embodiments, the collar includes one or more cut-outs or holes at or near the distal end that correspond in shape to end proximal connectors of the tethers. In some embodiments, the connectors have a rounded or mushroom shape, although it is appreciated the connectors and cut outs could be any suitable shape. Examples of these features are shown in FIGS. 34-38.

FIGS. 34A-34C depict various configurations of tethers 50 and interfacing of tethers within a retention collar 500, in accordance with some embodiments. FIG. 34A shows straight axially extending tethers 50a extending to a proximal connector 51. In this embodiment, the connector is a circular or rounded portion. FIG. 34B shows a tether 50b in which the proximal connector is a portion that curves inward to engage within a recess of a locking collar 500 of the delivery catheter, from which the tether can spring outward upon deployment to release the implant from the delivery catheter. FIG. 34C shows another tether 50c having sinusoidal portions that allow for axial stretch, as described previously, and that extend to a proximal connector 51 having a mushroom shape. It is appreciated that various other dimensions, shapes, and sizes could be used for the tether and proximal connectors.

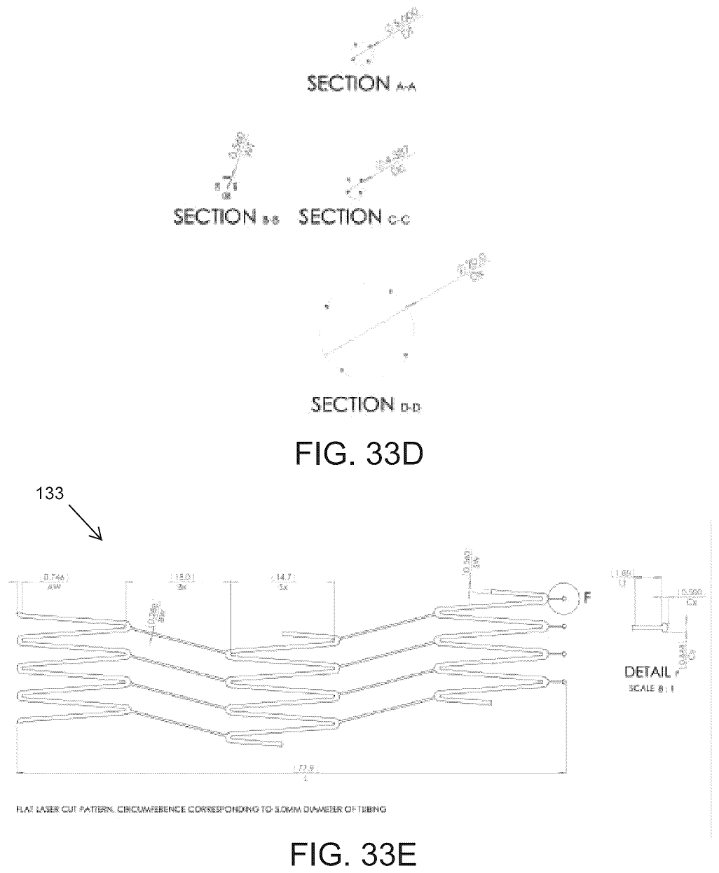

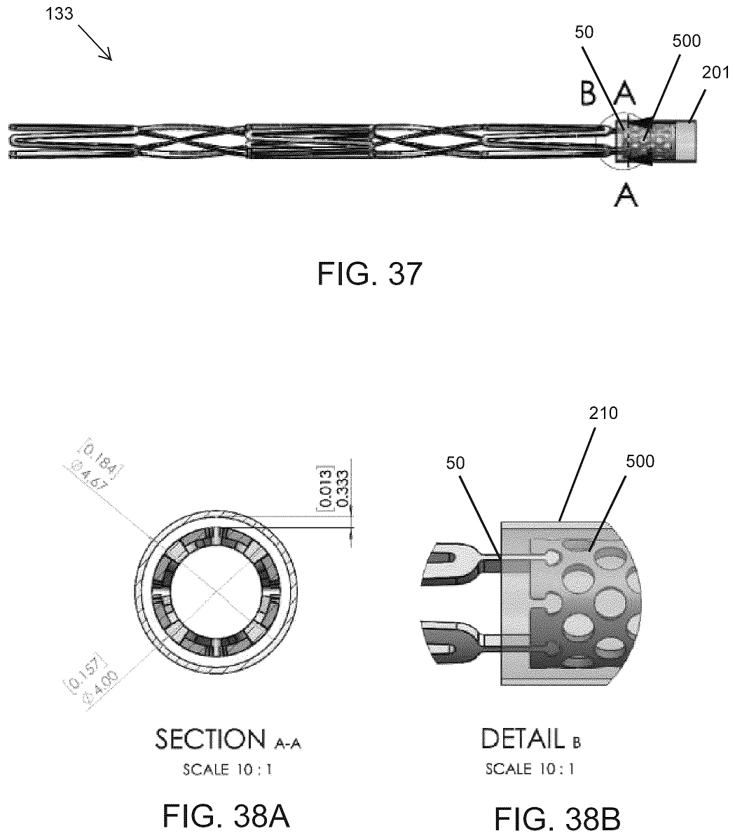

FIGS. 35A-35B depicts shorter tethers 50 that can be used for the 8-cell and 4-cell implant structures noted previously. In these embodiments, the tethers extend linearly and are relatively short (e.g., 1-10 mm, 2-6 mm, 2-4 mm) and extend to a proximal connector, which can also be referred to as a lock. In these embodiments, the proximal connector 51 can have a mushroom shape, although any suitable shape could be used. The proximal connector can be captured by a corresponding cut out within a retention collar disposed on a distal portion of the delivery catheter (as shown in FIGS. 38A-38B).

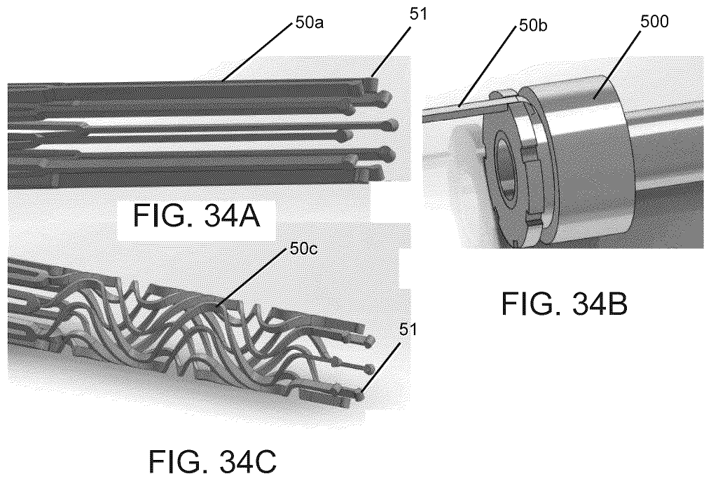

FIGS. 36A-36D depicts various views of a retention collar 500 for retaining the implant structure before full deployment, in accordance with some embodiments. The retention collar 500, which can also be referred to as a collar lock, can be laser cut, machined or formed by any suitable means. In this embodiment, the retention collar is formed from a metal tube (e.g., 4 ID Nitinol tube). The distal end include cut outs 501d correspond in shape to the proximal connector so as to secure the tether to the collar to facilitate control axial longitudinal movement of the implant when constrained within an outer sheath of the delivery catheter. The collar can further include an array of small holes (e.g., 1 mm holes) to facilitate adhesive bonding with the delivery shaft of the delivery catheter 200.

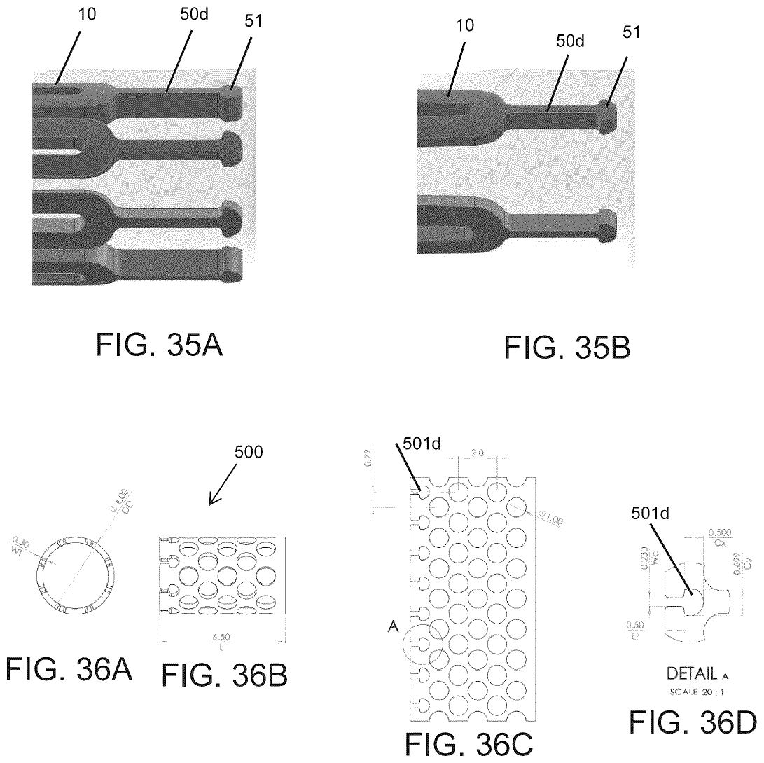

FIG. 37 depicts the implant 133 in the constrained configured extending from the outer shaft of the delivery catheter 200, where the retention tethers 50 are proximally engaged with the retention collar 500 attached to an advanceable delivery shaft of the delivery catheter. In some embodiments, this assembly can be fed through a 14 Fr outer shaft (i.e., introducer). Thus, by advancing the delivery shaft, the implant can be partially deployed (e.g., by maintaining the retention collar within the outer shaft), or fully deployed by advancing the collar beyond the distal end of the outer shaft to allow the proximal connectors to spring outward from the corresponding cut outs. This configuration allows the implant to be partially deployed, assessing of a baroreflex response, and then the implant can be repositioned or removed based on the response. FIGS. 38A-38B depict a cross-section view and a detail view of the proximal connectors of the tethers interfaced with the retention collar 500 where the implant is constrained within an outer sheath 210 of the delivery catheter, in accordance with some embodiments.

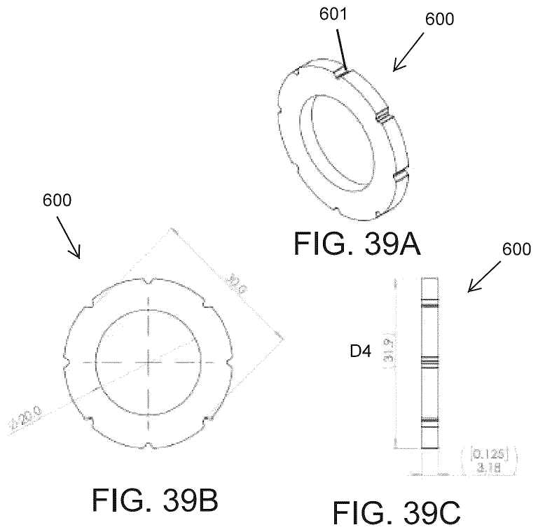

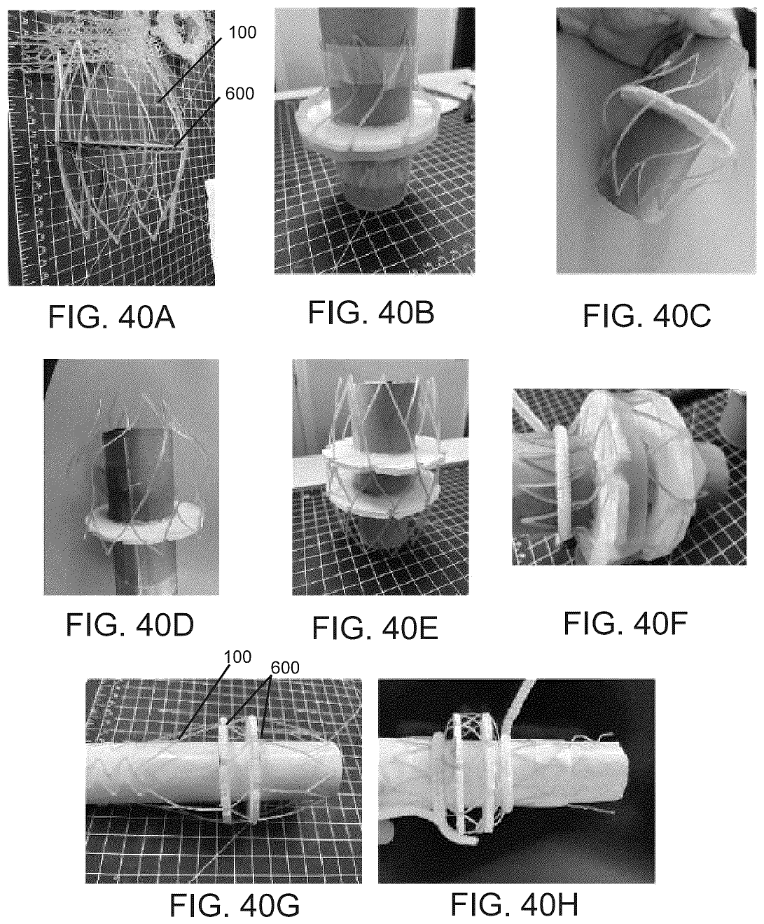

FIGS. 39A-39C depict a setting ring 600 for setting a diameter of the implant structure, in accordance with some embodiments. The setting ring 600 can include notches 601 to receive the struts of the expandable ring and the ring is dimensioned to hold and set the ring at the desired dimension sufficient to provide stretch at the target location. In this embodiments, notched rings can be cut from ⅛″ 304 stainless steel and positioned over a 20 mm mandrel. For implant embodiments having rings of varying diameters, multiple rings can be used, for example, one setting ring for 25 mm active center segment, and another setting ring for 30 mm active center segment. In a nominally 20 mm arch, these active segments provide 5 mm and 10 mm oversizing respectively. FIG. 40 depicts examples A-H of various implant structures being set to a specified diameter by use of one or more setting rings, in accordance with some embodiments.

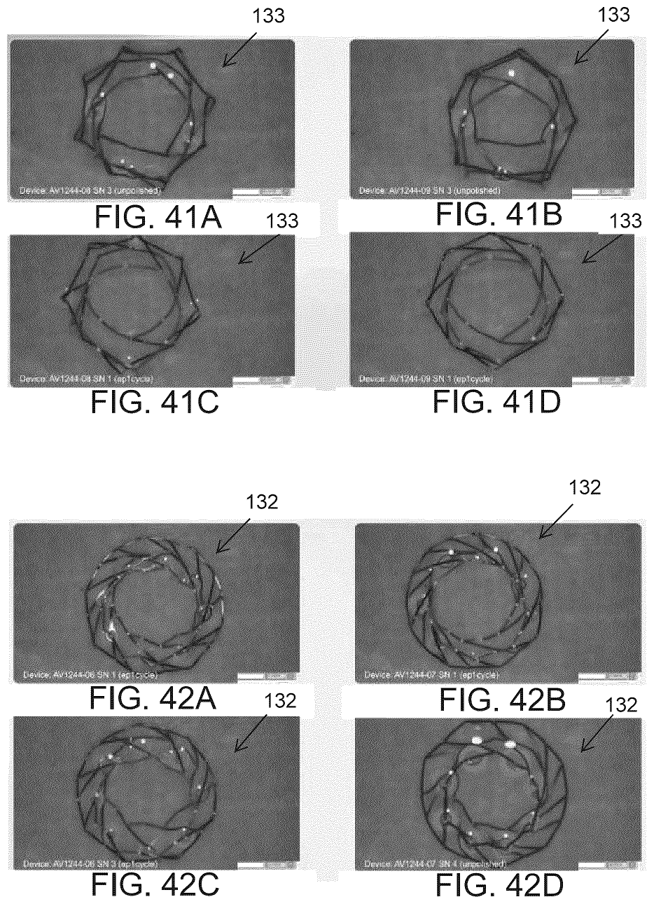

FIGS. 41A-42D illustrate cross-sections of the 4-cell and 8-cell expandable structures noted above for the all-in-one implant and baroreflex gauge. As shown in FIG. 41, the implant 133 having a 4-cell structure has a regular polygonal cross-section. As shown in FIG. 42, the implant 132 having a 8-cell structure has a substantially circular cross-section. Either design can be used to stretch the arterial wall by a suitable amount (e.g., 20% or more) for gauging of the baroreflex response and inducing the baroreflex response long-term after full deployment and implantation. In some embodiments, the implant may further include features to promote implantation, such as barbs, or features such as coatings, holes or slots to promote tissue-ingrowth. In some embodiments, these features may be disposed on a portion that is not yet deployed during gauging.

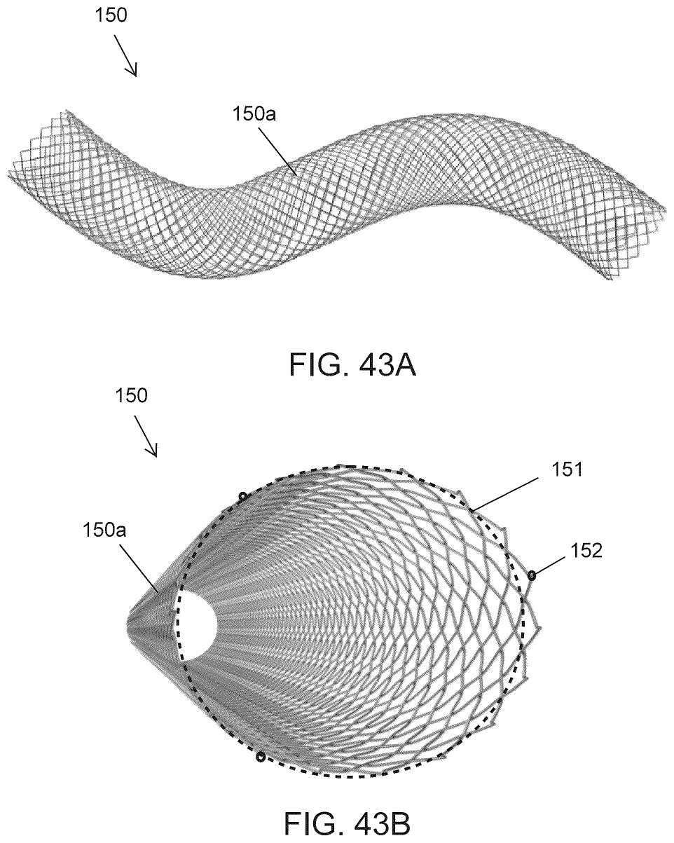

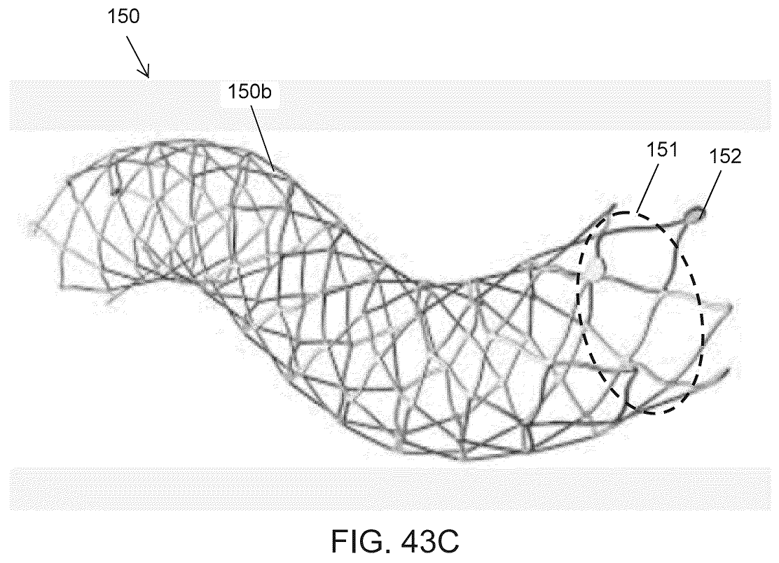

FIGS. 43A-43C depict another exemplary implant, a single expandable structure that spans across a substantial portion of the aortic arch, such as implant 150 shown in FIG. 1E. This implant is designed with a non-circular cross-sectional shape so as to change a shape of the aortic arch when deployed within. FIGS. 43A-43B shows the implant design 150a as a stent-like woven or braided expandable structure designed with a non-circular cross section when expanded, although the structure typically includes fewer braids/struts and larger openings, typically 30%, 40%, 50%, 60%, 70% fewer struts or less. As shown in FIG. 43A, the braided structure is sufficiently flexible in a lateral direction to accommodate the curvature of the aortic arch. As shown in FIG. 43B, the non-circular cross section 151 is elliptical. The implant can be formed of one or more wires that are braided such that the wires extend helically, which allows for greater lateral flexibility. FIG. 43C shows another implant design 150b that is formed as a laser-cut tube, the struts configured so that when deployed the implant forms the non-circular cross section 151 that is elliptical. In some embodiments, the implant can optionally include one or more coupling/release features 152 at one or both ends to facilitate delivery and deployment of the implant. Such features can include loops, cut-out portions, or any of those described herein.

In these embodiments, the implant 150 is at least 60 mm in length, typically about 70-90 mm, such that the implant extends along a majority of the aortic arch along the target zone, thereby activating multiple regions of baroreceptors and providing a more consistent, robust response. Moreover, by relying on engagement of the device along a majority of the aorta, the anchoring forces of the implant are distributed over a larger area avoiding the need for separate anchoring structures or features, thereby minimizing trauma to the arterial walls, which can reduce inflammation and formation of thrombus that can contribute to formation of atherosclerotic plaques.

It is understood that these concepts can be utilized in various other shapes/designs, the non-circular cross-section could be other non-circular shapes as well, including but not limited to: triangular, square, rectangular, or any regular polygonal shape.

In another aspect, the implant is sized specifically for the dimensions of the human aortic arch so as to engage the arterial walls with the structure so as to anchor the implant within the aortic arch and sufficiently engage the arterial walls within the target zone. Preferably, the target zone is sufficiently large to include multiple target regions or locations rich in sensitive baroreceptors, including the cylindrical band wrapping the aorta across and adjacent the LSA, as noted previously. Thus, engagement with a non-circular cross-section of a braided implant tensions the arterial wall and stimulates the highly sensitive baroreceptors in this region.

In yet another aspect, the implant device is especially suited for intravascular delivery and deployment since the implant has a collapsed configuration for advancement through the vasculature and an expanded configuration for engaging the arterial walls, as shown in the embodiments of FIGS. 1A-1E. In the collapsed configuration, the implant is disposed in a delivery catheter to facilitate intravascular delivery to the target site at the aortic arch and subsequent deployment.

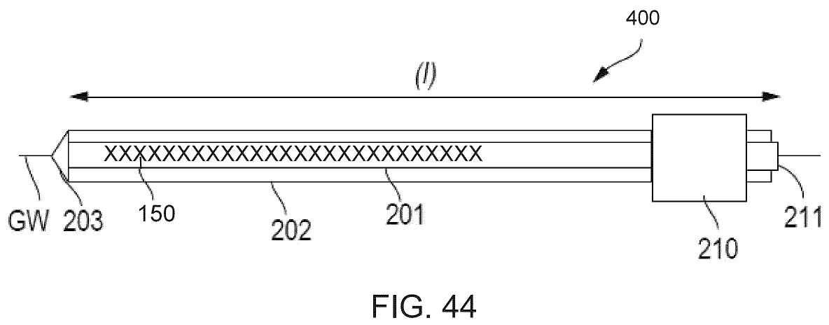

In an exemplary embodiment, the implant is a self-expandable structure that is preloaded into a sheathed delivery catheter, as shown in FIG. 44. As shown, the intravascular delivery catheter 400 is designed to deliver the implant 150 in the collapsed configuration, and to position and deploy the implant along the target zone within the aortic arch, as shown in FIGS. 1A-1D. The delivery catheter includes an internal guidewire lumen so that it can be advanced along a guidewire GW positioned in the aortic arch. In the embodiment shown, the delivery catheter 200 includes a catheter shaft 201 on which the implant 150 is collapsed, and over which is disposed a retractable sheath 202 that constrains the implant in the collapsed configuration until the implant is positioned at the desired target zone, for example by visualization of a marker (e.g., radiopaque or ultrasound marker). In some embodiments, the connectors may be made from a differing material than the frames so that the connectors themselves are distinctly visible through visualization techniques. The delivery catheter can further include a distal tip 203 to guide advancement over the GW and a flush port 211 for flushing before, during or after delivery. The delivery catheter includes a handle 210 by which the clinician can retract the sheath to deploy the self-expanding implant. Typically, the overall length ( ) of the delivery catheter is between 100-150 cm (e.g., about 135 cm) so as to readily access the aortic arch by insertion of the catheter through the femoral artery.

In some embodiments, the delivery catheter can be configured to deliver the entire implant upon retraction of the sheath. The length of the expandable structure is sufficient such that the expandable structure is deployed at the target zone despite any minor axial movement upon deployment. In some embodiments, the catheter may include incremental retraction to deliver the implant in a precise controlled manner. In still other embodiments, the implant may be balloon expandable and disposed in a collapsed configuration on a balloon of the delivery catheter, the balloon suitably dimensioned for expansion in the aorta to expand and deploy the implant in the target region.

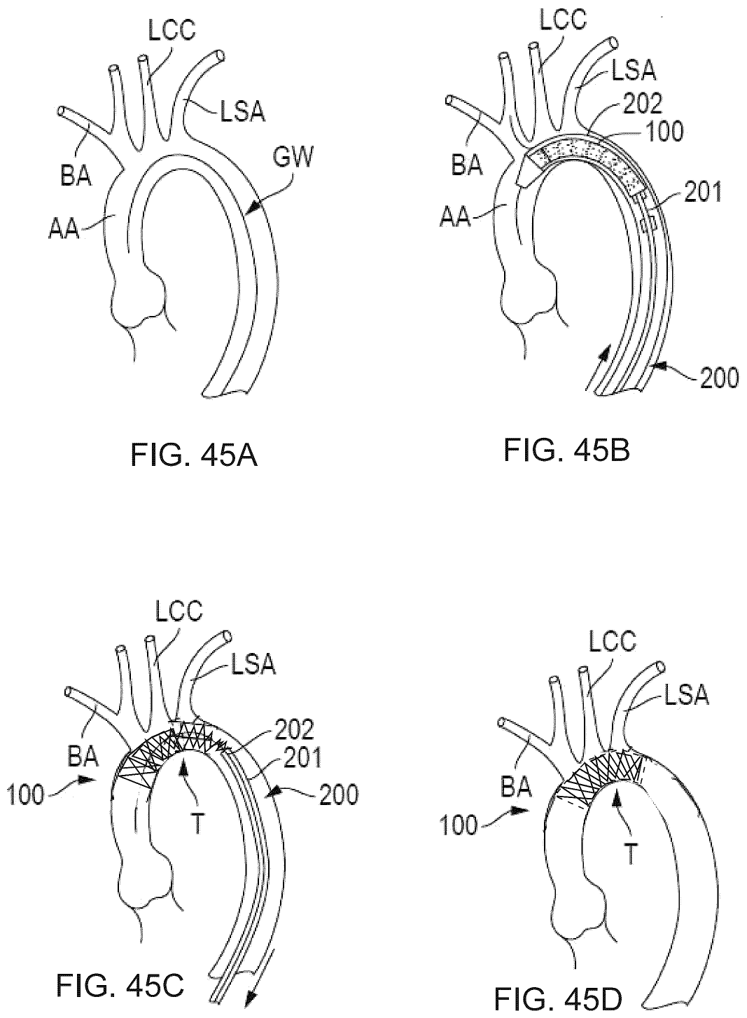

FIGS. 45A-45D illustrate sequential steps of an exemplary method of treating hypertension by deploying the implant device described herein. As shown in FIG. 45A, a guidewire GW is advanced through an entry point (e.g., the femoral artery) and advanced through the vasculature and into the aortic arch. Visualization techniques, such as fluoroscopy, can verify placement of the GW in the target region. As shown in FIG. 45B, the delivery catheter 200 is advanced along the GW, the catheter having an implant 100 disposed in a collapsed configuration on a catheter shaft 201 and constrained within a retractable outer sheath 202. Once the implant is positioned at the desired target location within the aortic arch, the outer sheath 202 is retracted, thereby allowing the self-expandable implant 100 to resilient deploy into its expanded configuration engaging the arterial walls, as shown in FIG. 45C. The guidewire GW and delivery catheter 201 are withdrawn, leaving the implant anchored at the target zone in the aortic arch for long-term reduction in blood pressure, as shown in FIG. 45D.

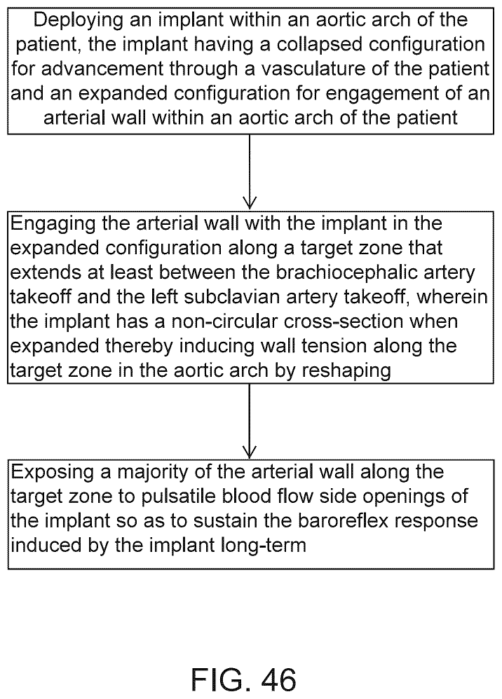

FIG. 46 pertains to a method of delivering an implant for treating hypertension. The method includes steps of: deploying an implant within an aortic arch of the patient, the implant having a collapsed configuration for advancement through a vasculature of the patient and an expanded configuration for engagement of an arterial wall within an aortic arch of the patient; engaging the arterial wall with the implant in the expanded configuration along a target zone that extends at least between the brachiocephalic artery takeoff and the left subclavian artery takeoff, wherein the implant has a non-circular cross-section when expanded thereby inducing wall tension along the target zone in the aortic arch by reshaping; and exposing a majority of the arterial wall along the target zone to pulsatile blood flow side openings of the implant so as to sustain the baroreflex response induced by the implant long-term.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art. Unless stated otherwise, the term “about” is considered to mean within +/−10%. It is appreciated that while various figures include dimensions or scale, that these are exemplary and the concepts herein are not limited to these dimensions. It is appreciated that various dimensions of embodiments described herein and that, in some embodiments, a respective dimension could encompass variations, such as within +/−25% or within +/−10% of the recited value. Any references to publication, patents, or patent applications are incorporated herein by reference in their entirety for all purposes.

1.-55. (canceled)

56. A method of delivering a hypertension treatment implant in a patient, said method comprising:
advancing a delivery catheter carrying an implant for treating hypertension in a distal portion thereof, the implant comprising one or more expandable structures and being disposed in a collapsed configuration on the delivery catheter to facilitate advancement through the vasculature of the patient, the entire implant, when deployed, being a length of 70 mm or more and having a non-circular cross-section;
positioning the distal portion of the catheter carrying the implant in the collapsed configuration at a target region within the aortic arch, wherein the target region includes a region extending between the left common carotid artery and the left subclavian artery takeoffs; and
deploying the implant within the aortic arch of the patient so that the implant assumes an expanded configuration so as to sufficiently engage at least a portion of the arterial wall along the target region, thereby triggering a baroreflex response of aortic arch baroreceptors within the target region to reduce blood pressure.

57. The method of claim 56, wherein positioning the distal portion of the catheter within the aortic arch comprises advancing the delivery catheter along a guidewire positioned within the aortic arch.

58. The method of claim 57, wherein positioning the distal portion of the catheter comprises observing one or more visualization markers disposed on the implant and/or the delivery catheter by visualization techniques.

59. The method of claim 57 wherein the target region includes an inner curvature of the aortic arch.

60. The method of claim 59 wherein positioning the implant at the target region comprises aligning the one or more visualization markers relative to the left subclavian artery extending from the aortic arch.

61. The method of claim 59 wherein positioning the implant at the target region comprises alignment of the one or more visualization markers with a second guidewire disposed within the left subclavian artery extending from the aortic arch.

62. The method of claim 56 the implant is self-expandable from the collapsed configuration to the expanded configuration, wherein the implant is constrained in the collapsed configuration by a retractable outer sheath of the delivery catheter.

63. The method of claim 62 wherein deploying the implant comprises retracting the outer sheath so as to release the self-expandable implant into the expanded configuration to engage the arterial wall in the target region.

64. The method of claim 56 wherein each expandable structure comprises one or more wires defining a design having a length greater than a lateral dimension thereof, wherein the implant is deployed such that the length of the expandable structure extends in a blood flow direction along the aorta.

65. The method of claim 56 further comprising:
withdrawing the delivery catheter, thereby leaving the deployed implant maintained at the target region to provide long term reduction in blood pressure.

66. The method of claim 56 further comprising:
inhibiting trauma of the arterial wall during deployment in the aortic arch by proximal and distal atraumatic ends of the implant.

67. A delivery catheter for deploying a hypertension treatment implant in a patient, the delivery catheter comprising:
a shaft extending between a proximal end and a distal end, the shaft having one or more lumens therethrough;
an implant disposed on a distal portion of the shaft in a collapsed configuration, wherein the implant is convertible between the collapsed configuration and an expanded configuration for engaging the arterial walls within the aortic arch, wherein the implant comprises one or more expandable structures sufficiently flexible in a lateral direction to accommodate a curvature of the aortic arch, wherein the an entire length of the implant, when deployed, is 70 mm or greater and having a non-circular cross section and positionable via the delivery catheter so as to engage a target region within the aortic arch;
a retractable outer sheath having a proximal end and a distal end and being disposed over the shaft including the distal end portion having the implant disposed thereon such that the delivery catheter is configured to facilitate deployment of the one or more expandable structures at the target region; and
a catheter handle disposed at or near the proximal end of the shaft, wherein the outer sheath is retractable from the catheter handle to facilitate deployment of the implant within the aortic arch with the at least one expandable structure of the plurality disposed at the target region in the aortic arch.

68. The delivery catheter of claim 67 wherein the target region includes a region extending between the left common carotid artery and the left subclavian artery.

69. The delivery catheter of claim 67 wherein the catheter is dimensioned for intravascular delivery and deployment of the implant to a target region through a femoral access site of the patient.

70. The delivery catheter of claim 67 wherein the catheter handle includes a retractable hub coupled to the outer shaft to allow manual retraction of the outer sheath for deployment of the implant.

71. The delivery catheter of claim 67 wherein the implant is self-expandable, wherein the outer sheath constrains the implant in the collapsed configuration during delivery.

72. The delivery catheter of claim 67 wherein the delivery catheter shaft and/or implant includes one or more visualization markers thereon to facilitate positioning of the implant at the target region within the aortic ach.

73. The delivery catheter of claim 72 wherein the one or more visualization markers are disposed on a portion of the implant.

74. The delivery catheter of claim 72 wherein the one or more visualization markers are disposed on a portion of the catheter shaft and/or the outer sheath.

75. The delivery catheter of claim 67 wherein the delivery catheter is configured to be advanced along a first guidewire disposed in the artic arch.

76. The delivery catheter of claim 67 wherein the outer sheath includes one or more markers thereon to facilitate retraction of the outer sheath a sufficient distance to deploy part or all of the implant.

77. The delivery catheter of claim 67 wherein the expandable structure comprises a one or more wires.
